(19) United States
(12) Patent Application Publication
(10) Pub. No.: US 2022/0062294 A1
(43) Pub. Date: Mar. 3, 2022

(54) PHARMACEUTICAL COMPOSITIONS OF TETRACYCLIC QUINOLONE ANALOGS AND THEIR SALTS

(71) Applicant: Senhwa Biosciences, Inc., New Taipei City (TW)

(72) Inventors: Hshiou-ting LIU, Milpitas, CA (US); John SOONG, Chino, CA (US)

(21) Appl. No.: 17/465,336

(22) Filed: Sep. 2, 2021

Related U.S. Application Data
(60) Provisional application No. 63/073,692, filed on Sep. 2, 2020.

(51) Int. Cl. A61K 31/551; A61K 9/08; A61K 47/02

(52) U.S. Cl. CPC A61K 31/551; A61K 47/02; A61K 9/08

(57) ABSTRACT

The present invention includes formulation comprising 2-(4-Methyl-[1,4]diazepan-1-yl)-5-oxo-5H-7-thia-1,11b-diaza-benzo[c]fluorene-6-carboxylic acid (5-methyl-pyrazin-2-ylmethyl)-amide (Compound I) or a pharmaceutically acceptable salt thereof for use in treating cancer.

### CROSS-REFERENCE TO RELATED APPLICATIONS

This application claims the priority benefit of U.S. Provisional Application No. 63/073,692, filed Sep. 2, 2020, the disclosures of which are incorporated by reference herein in their entireties for all purposes.

### FIELD OF THE DISCLOSURE

The present invention generally relates to fused tetracyclic quinolone analogs or a pharmaceutically acceptable salts thereof, pharmaceutical composition containing them, and methods of their use in treating diseases or disorders including cancer.

### BACKGROUND OF THE DISCLOSURE

A variety of tetracyclic quinolone compounds or napththyridinone fused tetracyclic compounds have been suggested to function by interacting with quadruplex-forming regions of nucleic acids and modulating ribosomal RNA transcription. See, for example, U.S. Pat. Nos. 7,928,100 and 8,853,234. Specifically, the tetracyclic quinolone compounds can stabilize the DNA G-quadruplexes (G4s) in cancer cells and thereby induce synthetic lethality in cancer cells. Since treatment of cells with G4-stabilizing agents can lead to the formation of DNA double strand breaks (DSBs), DSB formation induced by G4-stabilizing ligand/agent (such as the tetracyclic quinolones) treatment would be more pronounced in cells genetically deficient in, or chemically inhibited in, repair pathways including both non-homologous end joining (NHEJ) and homologous recombination (HR) repair. Furthermore, the tetracyclic quinolone compounds selectively inhibit rRNA synthesis by RNA polymerase I (Pol I) in the nucleolus, but do not inhibit mRNA synthesis by RNA polymerase II (Pol II) and do not inhibit DNA replication or protein synthesis. It is suggested that targeting RNA polymerase I (Pol I) to activate p53 through the nucleolar stress pathway may results in selective activation of p53 in tumor cells. The p53 protein normally functions as a tumor suppressor by causing cancer cells to self-destruct. Activating p53 to kill cancer cells is a well validated anticancer strategy and many approaches are being employed to exploit this pathway. Selective activation of p53 in tumor cells would be an attractive method of treating, controlling, ameliorating tumor cells while not affecting normal healthy cells. The aforementioned tetracyclic quinolones are disclosed in U.S. Pat. Nos. 7,928,100 and 8,853,234, and the contents of this publication are herein incorporated by reference in their entirety for all intended purposes.

### SUMMARY OF THE DISCLOSURE

In one embodiment, the present disclosure provides a liquid pharmaceutical composition comprising Compound I, or a pharmaceutically acceptable salt and/or solvate thereof and a pharmaceutically acceptable carrier or excipient,

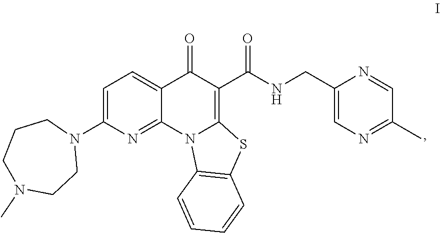

wherein the composition is substantially free of phosphates. In one embodiment, the composition comprises less than about 1% impurities. In one embodiment, the composition comprises less than about 0.5% impurities. In one embodiment, the composition comprises less than about 0.15% impurities.

In one embodiment, the present disclosure also provides a liquid pharmaceutical composition comprising Compound I, or a pharmaceutically acceptable salt and/or solvate thereof and a pharmaceutically acceptable carrier or excipient, wherein the composition comprises less than about 0.10% impurities.

In one embodiment of the liquid pharmaceutical compositions disclosed herein, the composition has a pH in the range of about 4.0 to about 6.5. In one embodiment of the liquid pharmaceutical compositions disclosed herein, the composition has a pH in the range of about 5.6 to about 6.0. In one embodiment, the composition has a pH of 5.8±0.1.

In one embodiment of the liquid pharmaceutical compositions disclosed herein, the composition is substantially free of aluminum salts, ions, or complexes. In one embodiment, the composition is substantially free of aluminophosphate. In one embodiment, the aluminophosphate has a chain of repeating [AlP₂O₈] units.

In one embodiment of the liquid pharmaceutical compositions disclosed herein, the composition is substantially free of a bulking agent. In one embodiment, the composition is substantially free of disaccharides or sugar alcohols. In one embodiment, the composition is substantially free of sucrose, mannitol, and trehalose.

In one embodiment of the liquid pharmaceutical compositions disclosed herein, the composition comprises sterile aqueous solution. In one embodiment, the composition comprises sterile saline solution. In one embodiment, the composition comprises 0.9% saline.

In one embodiment of the liquid pharmaceutical compositions disclosed herein, the composition comprises less than about 1 ppm of dissolved oxygen.

In one embodiment of the liquid pharmaceutical compositions disclosed herein, the composition comprises less than about 0.08% impurities. In one embodiment, the composition comprises less than about 0.07% impurities.

In one embodiment of the liquid pharmaceutical compositions disclosed herein, the composition comprises about 0.05% or less impurities after the composition is stored at a temperature in the range of about 2° C. to about 30° C. for 3 months. In one embodiment, the composition comprises about 0.06% or less impurities after the composition is stored at a temperature in the range of about 2° C. to about 30° C. for 6 months. In one embodiment, the composition comprises about 0.07% or less impurities after the composition is stored at a temperature in the range of about 2° C. to about 30° C. for 12 months. In one embodiment, the composition comprises about 0.07% or less impurities after the composition is stored at a temperature in the range of about 2° C. to about 30° C. for 18 months. In one embodiment, the composition comprises about 0.07% or less impurities after the composition is stored at a temperature in the range of about 2° C. to about 8° C. for 24 months. In one embodiment, the composition comprises about 0.12% or less impurities after the composition is stored at a temperature in the range of about 20° C. to about 30° C. for 24 months. In one embodiment, the impurity is Compound 7. In one embodiment, the composition is stored at a temperature of about 2° C. to about 8° C. In one embodiment, the composition is stored at a temperature of about 25° C./60% RH.

In one embodiment of the liquid pharmaceutical compositions disclosed herein, the composition is substantially free of Compound 1A. In one embodiment of the liquid pharmaceutical compositions disclosed herein, the composition is substantially free of Compound 10.

In one embodiment of the liquid pharmaceutical compositions disclosed herein, the composition is in a glass vial, a glass ampule or a glass container.

In one embodiment of the liquid pharmaceutical compositions disclosed herein, the composition has no visible precipitate or solid particulate.

In one embodiment of the liquid pharmaceutical compositions disclosed herein, the composition is substantially free of hydrated Compound I aluminophosphate complex.

In one embodiment of the liquid pharmaceutical compositions disclosed herein, the composition has been sparged with nitrogen. In one embodiment, the composition has been sparged with nitrogen to substantially remove dissolved oxygen. In one embodiment, the nitrogen sparged composition is substantially free of phosphate buffer. In one embodiment, the phosphate buffer is monosodium phosphate.

The present disclosure also provides a method for treating or ameliorating cell proliferation disorder in a subject, said method comprising administering to a subject in need thereof a therapeutically effective amount of any one of the liquid compositions as disclosed herein. In one embodiment, the cell proliferation disorder is cancer.

In one embodiment of the methods disclosed herein, cancer is heme cancer, colorectal cancer, breast cancer, lung cancer, liver cancer, ovarian cancer, cervical cancer, Ewing's sarcoma, pancreatic cancer, cancer of the lymph nodes, colon cancer, prostate cancer, brain cancer, cancer of the head and neck, bone cancer, skin cancer, kidney cancer, osteosarcoma, cancer of the heart, uterine cancer, gastrointestinal malignancies, and carcinomas of the larynx or oral cavity. In one embodiment, cancer is breast cancer, ovarian cancer, or pancreatic cancer. In one embodiment, heme cancer is leukemia, lymphoma, myeloma, or multiple myeloma.

In one embodiment of the methods disclosed herein, the subject has a mutation in a DNA repair gene. In one embodiment, the DNA repair gene is a homologous recombinant (HR) or non-homologous end joining (NHEJ) gene. In one embodiment, the DNA repair gene is a gene in the homologous recombination (HR) or non-homologous end joining (NHEJ) dependent deoxyribonucleic acid (DNA) double strand break (DSB) repair pathway.

In one embodiment of the methods disclosed herein, cancer is a BRCA-mutated or PALB2-mutated cancer. In one embodiment, cancer is BRCA2-mutated or BRCA1-mutated cancer. In one embodiment, cancer is characterized by one or more disease-associated mutations in BRCA1, BRCA2, or PALB2. In one embodiment, the mutation is a loss-of-function mutation. In one embodiment, the mutation is monoallelic loss-of-function mutation. In one embodiment, the mutation is biallelic loss-of-function mutation. In one embodiment, cancer is breast cancer, ovarian cancer, pancreatic cancer, or prostate cancer.

The present disclosure also provides a method of inhibiting Pol I transcription in a subject, comprising administering to a subject in need thereof a therapeutically effective amount of any one of the liquid compositions as disclosed herein. In one embodiment, inhibiting Pol I transcription is in peripheral blood mononuclear cells.

The present disclosure also provides a method of stabilizing G-quadruplexes (G4s) in a subject, comprising administering to a subject in need thereof a therapeutically effective amount of any one of the liquid compositions as disclosed herein. In one embodiment, the stabilizing G4s is in peripheral blood mononuclear cells.

In one embodiment of the methods disclosed herein, the liquid composition is administered intravenously.

### BRIEF DESCRIPTION OF THE DRAWINGS

FIG. 1 shows best % tumor shrinkage from baseline in all patients with BRCA1 or BRCA2 mutation from study described in Example 6.

FIG. 2 shows best % tumor shrinkage from baseline in all breast cancer patients with BRCA1 or BRCA2 mutation in patients from study described in Example 6.

FIG. 3 shows best % tumor shrinkage from baseline in all breast cancer patients with BRCA2 mutation from study described in Example 6.

FIG. 4 shows packing diagram of hydrated Compound I aluminophosphate viewed down the crystallographic b axis.

### DETAILED DESCRIPTIONS OF THE DISCLOSURE

The present invention relates to 2-(4-Methyl-[1,4]diazepan-1-yl)-5-oxo-5H-7-thia-1,11b-diaza-benzo[c]fluorene-6-carboxylic acid (5-methyl-pyrazin-2-ylmethyl)-amide (Compound I) or a pharmaceutically acceptable salts or solvates thereof. Compound I or a pharmaceutically acceptable salts or solvates thereof can stabilize G-quadruplexes (G4s) and/or inhibit Pol I and can be useful for treating disorders characterized by proliferation of cells.

### Definitions

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

Following long-standing patent law convention, the terms “a”, “an”, and “the” refer to “one or more” when used in this application, including the claims. Thus, for example, reference to “a carrier” includes mixtures of one or more carriers, two or more carriers, and the like.

The term “compound(s) of the present invention” or “compound(s) of the present disclosure” refers to 2-(4-Methyl-[1,4]diazepan-1-yl)-5-oxo-5H-7-thia-1,11b-diaza-benzo[c]fluorene-6-carboxylic acid (5-methyl-pyrazin-2-ylmethyl)-amide (Compound I) or isomers, salts, N-oxides, sulfoxides, sulfones, or solvates thereof.

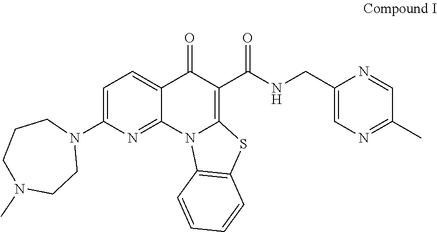

The term “isomer” refers to compounds having the same chemical formula but may have different stereochemical formula, structural formula, or special arrangements of atoms. Examples of isomers include stereoisomers, diastereomers, enantiomers, conformational isomers, rotamers, geometric isomers, and atropisomers.

The term “ester” refers to any ester of a compound of the present invention in which any of the —COOH functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The term “ester” includes but is not limited to pharmaceutically acceptable esters thereof. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids.

The term “room temperature” as used herein, means from 21 degrees Celsius to 27 degrees Celsius.

The term “composition” denotes one or more substance in a physical form, such as solid, liquid, gas, or a mixture thereof. One example of composition is a pharmaceutical composition, i.e., a composition related to, prepared for, or used in medical treatment. The term “formulation” is also used to indicate one or more substance in a physical form, such as solid, liquid, gas, or a mixture thereof.

The term “co-administration” or “coadministration” refers to administration of a formulation or a composition comprising Compound I, or a pharmaceutically acceptable salt or solvate thereof; and (b) one or more additional therapeutic agent and/or radio therapy, in combination, i.e., together in a coordinated fashion.

The term “carboxylic acid” refers to an organic acid characterized by one or more carboxyl groups, such as acetic acid and oxalic acid. “Sulfonic acid” refers to an organic acid with the general formula of R—(S(O)₂—OH)ₙ, wherein R is an organic moiety and n is an integer above zero, such as 1, 2, and 3. The term “polyhydroxy acid” refers to a carboxylic acid containing two or more hydroxyl groups. Examples of polyhydroxy acid include, but are not limited to, lactobionic acid, gluconic acid, and galactose.

As used herein, “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

“Salts” include derivatives of an active agent, wherein the active agent is modified by making acid or base addition salts thereof. Preferably, the salts are pharmaceutically acceptable salts. Such salts include, but are not limited to, pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine dicyclohexylamine and the like. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Standard methods for the preparation of pharmaceutically acceptable salts and their formulations are well known in the art, and are disclosed in various references, including for example, “Remington: The Science and Practice of Pharmacy”, A. Gennaro, ed., 20th edition, Lippincott, Williams & Wilkins, Philadelphia, Pa.

As used herein, “solvate” means a complex formed by solvation (the combination of solvent molecules with molecules or ions of the compounds of the present invention), or an aggregate that consists of a solute ion or molecule (the compounds of the present invention) with one or more solvent molecules. In the present invention, the preferred solvate is hydrate. Examples of hydrate include, but are not limited to, hemihydrate, monohydrate, dihydrate, trihydrate, hexahydrate, etc. It should be understood by one of ordinary skill in the art that the pharmaceutically acceptable salt of the present compound may also exist in a solvate form. The solvate is typically formed via hydration which is either part of the preparation of the present compound or through natural absorption of moisture by the anhydrous compound of the present invention. Solvates including hydrates may be consisting in stoichiometric ratios, for example, with two, three, four salt molecules per solvate or per hydrate molecule. Another possibility, for example, that two salt molecules are stoichiometric related to three, five, seven solvent or hydrate molecules. Solvents used for crystallization, such as alcohols, especially methanol and ethanol; aldehydes; ketones, especially acetone; esters, e.g. ethyl acetate; may be embedded in the crystal grating. Preferred are pharmaceutically acceptable solvents.

The term “substantially similar” as used herein with regards to bioavailability of pharmacokinetics means that the two or more therapeutically active agents or drugs provide the same therapeutic effects in a subject.

The term “substantially free of” as used herein, means absence, undetectable, trace amounts, or present in de minimis amount of compounds or salts in a pharmaceutical composition. De minimis amount of compounds or salts in a pharmaceutical composition does not alter the stability of the composition.

The terms “excipient”, “carrier”, and “vehicle” are used interchangeably throughout this application and denote a substance with which a compound of the present invention is administered.

“Therapeutically effective amount” means the amount of a therapeutically active agent, when administered to a patient for treating a disease or other undesirable medical condition, is sufficient to have a beneficial effect with respect to that disease or condition. The therapeutically effective amount will vary depending on the therapeutically active agent, the disease or condition and its severity, and the age, weight, etc. of the patient to be treated. Determining the therapeutically effective amount of the therapeutically active agent is within the ordinary skill of the art and requires no more than routine experimentation.

As used herein, the terms “additional pharmaceutical agent” or “additional therapeutic agent” or “additional therapeutically active agent” with respect to the compounds described herein refers to an active agent other than the Compound I or a pharmaceutically acceptable salt or solvate thereof, which is administered to elicit a therapeutic effect. The pharmaceutical agent(s) may be directed to a therapeutic effect related to the condition that the compounds of the present disclosure is intended to treat or ameliorate (e.g., cancer) or, the pharmaceutical agent may be intended to treat or ameliorate a symptom of the underlying condition (e.g., tumor growth, hemorrhage, ulceration, pain, enlarged lymph nodes, cough, jaundice, swelling, weight loss, cachexia, sweating, anemia, paraneoplastic phenomena, thrombosis, etc.) or to further reduce the appearance or severity of side effects of the compounds of the present disclosure.

As used herein, the phrase “a disorder characterized by cell proliferation” or “a condition characterized by cell proliferation” include, but are not limited to, cancer, benign and malignant tumors. Examples of cancer and tumors include, but are not limited to, cancers or tumor growth of the colorectum, breast (including inflammatory breast cancer), lung, liver, pancreas, lymph node, colon, prostate, brain, head and neck, skin, kidney, osteosarcoma, blood and heart (e.g., leukemia, lymphoma, and carcinoma).

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “patient” or “subject” as used herein, includes humans and animals, preferably mammals.

As used herein, the terms “inhibiting” or “reducing” cell proliferation is meant to slow down, to decrease, or, for example, to stop the amount of cell proliferation, as measured using methods known to those of ordinary skill in the art, by, for example, 10%, 20%, 30%, 40%, 50%, 60%, 70%, 80%, 90%, 95%, or 100%, when compared to proliferating cells that are not subjected to the methods and compositions of the present application.

As used herein, the term “apoptosis” refers to an intrinsic cell self-destruction or suicide program. In response to a triggering stimulus, cells undergo a cascade of events including cell shrinkage, blebbing of cell membranes and chromatic condensation and fragmentation. These events culminate in cell conversion to clusters of membrane-bound particles (apoptotic bodies), which are thereafter engulfed by macrophages.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present application.

Compound I

Compound I is a synthetically derived small molecule, which can selectively binds and stabilizes DNA G-quadruplex (G4) structures. Key attributes of Compound I include induction of DNA damage through G4 stabilization which is dependent on intact BRCA1/2 and other homologous recombination mediated pathways for resolution. Cumulative mutations affecting BRCA1/2 and homologous recombination (HR) deficient tumor cells result in synthetic lethality.

Compound I showed specific toxicity against BRCA1/2 deficient cells in a number of cell lines of different genetic backgrounds (colon, breast and ovary) and different specifies origin (mouse and human). Compound I exhibited a wide therapeutic index of activity in BRCA2 knockout tumor cells in a xenograft model, when compared with isogenic wild type control cells. Without bound to any theory, the data to date attribute the anti-tumor activity of Compound I to bind and stabilize G4 DNA structure and impede the progression of DNA replication complexes and induces single stranded DNA gaps or breaks. The BRCA pathway is required for the repair of Compound I induced DNA damage, and that compromised DNA damage repair in the absence of BRCA genes will lead to lethality. BRCA deficient cells can be killed by Compound I at low drug concentration which are not effective at inhibiting rDNA transcription which suggests, without bound to any theory, that the dose used to treat BRCA deficient cancers is lower than that required to inhibit RNA Polymerase I and disrupt nucleons function.

Further, Compound I has shown to be responsive to PALB2 mutated cancers. The PALB2 gene is called the partner and localizer of the BRCA2 gene. It provides instructions to make a protein that works with the BRCA2 protein to repair damaged DNA and stop tumor growth. Inheriting two abnormal PALB2 genes causes Fanconi anemia type N, which suppresses bone marrow function and leads to extremely low levels of red blood cells, white blood cells, and platelets.

In some embodiments, Compound I is a free base. In other embodiments, Compound I is provided as a pharmaceutically acceptable salt. In one embodiment, the salt is a hydrochloric acid addition salt, a sulfuric acid addition salt, a sulfonic acid addition salt, a carboxylic acid addition salt, or a polyhydroxy acid addition salt. In one embodiment, Compound I is a hydrochloric acid salt.

Compound I exhibited antiproliferation activity against a variety of cancer cell lines. See Example 5.

Pharmaceutical Formulations

In one embodiment, the present invention provides a pharmaceutical composition comprising a therapeutically effective amount of a Compound I, or a pharmaceutically acceptable salt, ester, and/or solvate thereof, as disclosed herein, as the active ingredient, combined with a pharmaceutically acceptable excipient or carrier. The excipients are added to the formulation for a variety of purposes.

The present disclosure also relates to a liquid formulation comprising Compound I, or a pharmaceutically acceptable salt, ester, and/or solvate thereof, as disclosed herein.

In one embodiment, the formulation comprising Compound I, or a pharmaceutically acceptable salt, ester, and/or solvate thereof, has a pH from about 4.0 to about 6.5. In one embodiment, the formulation comprising Compound I, or a pharmaceutically acceptable salt, ester, and/or solvate thereof, has a pH from about 4.0 to about 6.0. In one embodiment, the formulation comprising Compound I, or a pharmaceutically acceptable salt, ester, and/or solvate thereof, has a pH from about 5.0 to about 6.0. In one embodiment, the formulation comprising Compound I, or a pharmaceutically acceptable salt, ester, and/or solvate thereof, has a pH from about 5.6 to about 6.0. In one embodiment, the formulation comprising Compound I, or a pharmaceutically acceptable salt, ester, and/or solvate thereof, has a pH of about 5.5, about 5.6, about 5.7, about 5.8, about 5.9, or about 6.0. In one embodiment, the formulation comprising Compound I, or a pharmaceutically acceptable salt, ester, and/or solvate thereof, has a pH of about 5.8. In one embodiment, the composition has a pH of 5.8±0.1.

In one embodiment, the formulation comprising Compound I, or a pharmaceutically acceptable salt, ester, and/or solvate thereof, pH of the formulation is adjusted with aqueous acid and aqueous base. In one embodiment, the formulation comprising Compound I, or a pharmaceutically acceptable salt, ester, and/or solvate thereof, pH of the formulation is adjusted with aqueous hydrochloric acid and aqueous sodium hydroxide. In one embodiment, the formulation comprising Compound I, or a pharmaceutically acceptable salt, ester, and/or solvate thereof, pH of the formulation is adjusted with 1N HCl and 1N NaOH.

In one embodiment, the formulation comprising Compound I, or a pharmaceutically acceptable salt, ester, and/or solvate thereof, comprises sterile solution. In one embodiment, the formulation comprising Compound I, or a pharmaceutically acceptable salt, ester, and/or solvate thereof, comprises water, glucose solution, dextrose solution, sucrose solution, or saline solution. In one embodiment, the formulation comprising Compound I, or a pharmaceutically acceptable salt, ester, and/or solvate thereof, comprises 0.9% saline solution.

In one embodiment, the formulation comprising Compound I, or a pharmaceutically acceptable salt, ester, and/or solvate thereof, comprises less than about 1 ppm of dissolved oxygen. In one embodiment of the preparation of any one of the formulations as disclosed herein, the dissolved oxygen content was maintained below 1 ppm. In one embodiment of the preparation of any one of the formulations as disclosed herein, the dissolved oxygen content was maintained below 1 ppm by sparging nitrogen to the formulation and/or solutions used in preparation of the formulation. In one embodiment of the preparation of any one of the formulations as disclosed herein, the dissolved oxygen content was maintained below 1 ppm by preparing the formulation under anaerobic conditions, such as inside a glove box.

In one embodiment, the formulation comprising Compound I, or a pharmaceutically acceptable salt, ester, and/or solvate thereof, is substantially free of bulking agent. In one embodiment, the formulation comprising Compound I, or a pharmaceutically acceptable salt, ester, and/or solvate thereof, is substantially free of sucrose, mannitol, or trehalose. In one embodiment, the formulation comprising Compound I, or a pharmaceutically acceptable salt, ester, and/or solvate thereof, is substantially free of disaccharide sugars. In one embodiment, the formulation comprising Compound I, or a pharmaceutically acceptable salt, ester, and/or solvate thereof, is substantially free of sugar alcohols. In some embodiments, the formulation disclosed herein that is substantially free of disaccharides and/or sugar alcohols is more stable than formulation comprising Compound I, or a pharmaceutically acceptable salt, ester, and/or solvate thereof, and disaccharides or sugar alcohols.

In one embodiment, the formulation comprising Compound I, or a pharmaceutically acceptable salt, ester, and/or solvate thereof, is substantially free of phosphates. In one embodiment, the formulation comprising Compound I, or a pharmaceutically acceptable salt, ester, and/or solvate thereof, is substantially free of phosphate buffers. In one embodiment, the formulation comprising Compound I, or a pharmaceutically acceptable salt, ester, and/or solvate thereof, is substantially free of monobasic sodium phosphate or dibasic sodium phosphate.

In one embodiment, the formulation comprising Compound I, or a pharmaceutically acceptable salt, ester, and/or solvate thereof, is substantially free of aluminophosphates. In one embodiment, the formulation comprising Compound I, or a pharmaceutically acceptable salt, ester, and/or solvate thereof, is substantially free of aluminophosphate having a chain of repeating [AlP₂O₈] units. In one embodiment, the formulation comprising Compound I, or a pharmaceutically acceptable salt, ester, and/or solvate thereof, is substantially free of aluminum salts, ions, or complexes. In one embodiment, the formulation comprising Compound I, or a pharmaceutically acceptable salt, ester, and/or solvate thereof, is substantially free of phosphate salts, ions, or complexes.

In one embodiment, the formulation comprising Compound I, or a pharmaceutically acceptable salt, ester, and/or solvate thereof, is substantially free of Compound I aluminophophate complex.

In one embodiment, the formulation comprising Compound I, or a pharmaceutically acceptable salt, ester, and/or solvate thereof, comprises less than about 1%, less than about 0.5%, less than about 0.4%, less than about 0.3%, less than about 0.2%, less than about 0.1%, less than about 0.09%, less than about 0.08%, less than about 0.07%, less than about 0.06%, or less than about 0.05% impurities, including all values therebetween.

In one embodiment, the formulation comprising Compound I, or a pharmaceutically acceptable salt, ester, and/or solvate thereof, comprises less than about 1%, less than about 0.5%, less than about 0.4%, less than about 0.3%, less than about 0.2%, less than about 0.1%, less than about 0.09%, less than about 0.08%, less than about 0.07%, less than about 0.06%, or less than about 0.05% impurities after the formulation is stored at 2-8° C. or at about 25° C./60% RH for 1 month or 3 months, including all values therebetween.

In one embodiment, the formulation comprising Compound I, or a pharmaceutically acceptable salt, ester, and/or solvate thereof, comprises less than about 1%, less than about 0.5%, less than about 0.4%, less than about 0.3%, less than about 0.2%, less than about 0.1%, less than about 0.09%, less than about 0.08%, less than about 0.07%, less than about 0.06%, or less than about 0.05% impurities after the formulation is stored at 2-8° C. or at about 25° C./60% RH for 6 months or 9 months, including all values therebetween.

In one embodiment, the formulation comprising Compound I, or a pharmaceutically acceptable salt, ester, and/or solvate thereof, comprises less than about 1%, less than about 0.5%, less than about 0.4%, less than about 0.3%, less than about 0.2%, less than about 0.1%, less than about 0.09%, less than about 0.08%, or less than about 0.07% impurities after the formulation is stored at 2-8° C. or at about 25° C./60% RH for 12 months, including all values therebetween.

In one embodiment, the formulation comprising Compound I, or a pharmaceutically acceptable salt, ester, and/or solvate thereof, comprises less than about 1%, less than about 0.5%, less than about 0.4%, less than about 0.3%, less than about 0.2%, less than about 0.1%, less than about 0.09%, less than about 0.08%, less than about 0.07%, less than about 0.06%, or less than about 0.05% impurities after the formulation is stored at 2-8° C. for 18 months, including all values therebetween. In one embodiment, the formulation comprising Compound I, or a pharmaceutically acceptable salt, ester, and/or solvate thereof, comprises less than about 0.07% impurities after the formulation is stored at 2-8° C. for 18 months.

In one embodiment, the formulation comprising Compound I, or a pharmaceutically acceptable salt, ester, and/or solvate thereof, comprises less than about 1%, less than about 0.5%, less than about 0.4%, less than about 0.3%, less than about 0.2%, less than about 0.1%, less than about 0.09%, less than about 0.08%, less than about 0.07%, less than about 0.06%, or less than about 0.05% impurities after the formulation is stored at about 25° C./60% RH for 18 months, including all values therebetween. In one embodiment, the formulation comprising Compound I, or a pharmaceutically acceptable salt, ester, and/or solvate thereof, comprises less than about 0.07% impurities after the formulation is stored at about 25° C./60% RH for 18 months.

In one embodiment, the formulation comprising Compound I, or a pharmaceutically acceptable salt, ester, and/or solvate thereof, comprises less than about 1%, less than about 0.5%, less than about 0.4%, less than about 0.3%, less than about 0.2%, less than about 0.1%, less than about 0.09%, less than about 0.08%, or less than about 0.07%, impurities after the formulation is stored at 2-8° C. for 24 months, including all values therebetween. In one embodiment, the formulation comprising Compound I, or a pharmaceutically acceptable salt, ester, and/or solvate thereof, comprises less than about 0.07% impurities after the formulation is stored at 2-8° C. for 24 months. In one embodiment, the formulation comprising Compound I, or a pharmaceutically acceptable salt, ester, and/or solvate thereof, comprises less than about 0.05% impurities after the formulation is stored at 2-8° C. for 24 months.

In one embodiment, the formulation comprising Compound I, or a pharmaceutically acceptable salt, ester, and/or solvate thereof, comprises less than about 1%, less than about 0.5%, less than about 0.4%, less than about 0.3%, less than about 0.2%, less than about 0.19%, less than about 0.18%, less than about 0.17%, less than about 0.16%, less than about 0.15%, less than about 0.14%, less than about 0.13%, less than about 0.12%, less than about 0.11%, less than about 0.10%, less than about 0.09%, less than about 0.08%, or less than about 0.07%, impurities after the formulation is stored at about 25° C./60% RH for 24 months, including all values therebetween. In one embodiment, the formulation comprising Compound I, or a pharmaceutically acceptable salt, ester, and/or solvate thereof, comprises less than about 0.15% impurities after the formulation is stored at about 25° C./60% RH for 24 months. In one embodiment, the formulation comprising Compound I, or a pharmaceutically acceptable salt, ester, and/or solvate thereof, comprises less than about 0.12% impurities after the formulation is stored at about 25° C./60% RH for 24 months.

In one embodiment, the formulation comprising Compound I, or a pharmaceutically acceptable salt, ester, and/or solvate thereof, comprises less than about 1%, less than about 0.5%, less than about 0.4%, less than about 0.3%, less than about 0.2%, less than about 0.15%, less than about 0.1%, less than about 0.09%, less than about 0.08%, or less than about 0.07% impurities after the formulation is stored at 2-8° C. or at about 25° C./60% RH for 18 months, including all values therebetween. In one embodiment, the formulation comprising Compound I, or a pharmaceutically acceptable salt, ester, and/or solvate thereof, comprises less than about 1%, less than about 0.5%, less than about 0.4%, less than about 0.3%, less than about 0.2%, less than about 0.15%, less than about 0.1%, less than about 0.09%, less than about 0.08%, or less than about 0.07% impurities after the formulation is stored at 2-8° C. or at about 25° C./60% RH for 24 months, including all values therebetween.

In one embodiment, the formulation comprising Compound I, or a pharmaceutically acceptable salt, ester, and/or solvate thereof, comprises less than about 1%, less than about 0.5%, less than about 0.4%, less than about 0.3%, less than about 0.2%, less than about 0.15%, less than about 0.1%, less than about 0.09%, less than about 0.08%, less than about 0.07%, less than about 0.06%, or less than about 0.05% Compound 1A, including all values therebetween. In one embodiment, the formulation comprises less than about 0.1%, less than about 0.09%, less than about 0.08%, less than about 0.07%, less than about 0.06%, or less than about 0.05% Compound 1A, including all values therebetween. In one embodiment, the formulation is substantially free of Compound 1A.

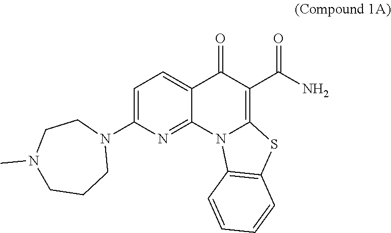 [SMILES: CN1CCCN(c2ccc3c(=O)c(C(N)=O)c4sc5ccccc5n4c3n2)CC1]

In one embodiment, the formulation comprising Compound I, or a pharmaceutically acceptable salt, ester, and/or solvate thereof, comprises less than about 1%, less than about 0.5%, less than about 0.4%, less than about 0.3%, less than about 0.2%, less than about 0.15%, less than about 0.1%, less than about 0.09%, less than about 0.08%, less than about 0.07%, less than about 0.06%, or less than about 0.05% Compound 7, including all values therebetween. In one embodiment, the formulation comprises less than about 0.1%, less than about 0.09%, less than about 0.08%, less than about 0.07%, less than about 0.06%, or less than about 0.05% Compound 7, including all values therebetween.

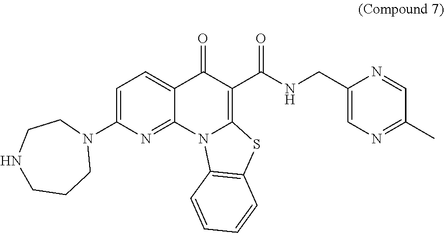 [SMILES: Cc1cnc(CNC(=O)c2c(=O)c3ccc(N4CCCNCC4)nc3n3c2sc2ccccc23)cn1]

In one embodiment, the formulation comprising Compound I, or a pharmaceutically acceptable salt, ester, and/or solvate thereof, comprises less than about 1%, less than about 0.5%, less than about 0.4%, less than about 0.3%, less than about 0.2%, less than about 0.15%, less than about 0.1%, less than about 0.09%, less than about 0.08%, less than about 0.07%, less than about 0.06%, or less than about 0.05% Compound 10, including all values therebetween. In one embodiment, the formulation comprises less than about 0.1%, less than about 0.09%, less than about 0.08%, less than about 0.07%, less than about 0.06%, or less than about 0.05% Compound 10, including all values therebetween. In one embodiment, the formulation is substantially free of Compound 10.

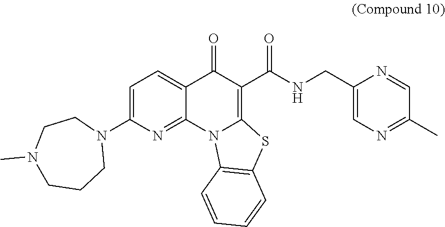

In one embodiment, the formulation is substantially free of Compound 10 after the formulation is stored at about 2° C. to about 30° C. for 1 month, 3 months, 6 months, 9 months, 12 months, or 18 months. In one embodiment, the formulation is substantially free of Compound 10 after the formulation is stored at 2-8° C. or at about 25° C./60% RH for 1 month, 3 months, 6 months, 9 months, 12 months, or 18 months.

In one embodiment, the formulation comprising Compound I, or a pharmaceutically acceptable salt, ester, and/or solvate thereof, comprises less than about 1%, less than about 0.5%, less than about 0.4%, less than about 0.3%, less than about 0.2%, less than about 0.15%, less than about 0.1%, less than about 0.09%, less than about 0.08%, less than about 0.07%, less than about 0.06%, or less than about 0.05% Compound 10 after the formulation is stored at 2-8° C. or at about 25° C./60% RH for 1 month, 3 months, 6 months, 9 months, 12 months, 18 months, or 24 months, including all values therebetween.

In one embodiment, the formulation comprising Compound I, or a pharmaceutically acceptable salt, ester, and/or solvate thereof, comprises less than about 0.05% Compound 10 after the formulation is stored at 2-8° C. for 1 month, 3 months, 6 months, 9 months, 12 months, 18 months, or 24 months, including all values therebetween.

In one embodiment, the formulation comprising Compound I, or a pharmaceutically acceptable salt, ester, and/or solvate thereof, comprises less than about 0.05% Compound 10 after the formulation is stored at about 25° C./60% RH for 1 month, 3 months, 6 months, 9 months, 12 months, or 18 months, including all values therebetween. In one embodiment, the formulation comprising Compound I, or a pharmaceutically acceptable salt, ester, and/or solvate thereof, comprises less than about 1%, less than about 0.5%, less than about 0.4%, less than about 0.3%, less than about 0.2%, less than about 0.15%, less than about 0.1%, less than about 0.09%, less than about 0.08%, less than about 0.07%, or less than about 0.06% Compound 10 after the formulation is stored at about 25° C./60% RH for about 24 months. In one embodiment, the formulation comprising Compound I, or a pharmaceutically acceptable salt, ester, and/or solvate thereof, comprises less than about 0.06% Compound 10 after the formulation is stored at about 25° C./60% RH for 24 months.

In one embodiment, the formulation comprising Compound I, or a pharmaceutically acceptable salt, ester, and/or solvate thereof, comprises less than about 1%, less than about 0.5%, less than about 0.4%, less than about 0.3%, less than about 0.2%, less than about 0.15%, less than about 0.1%, less than about 0.09%, less than about 0.08%, less than about 0.07%, less than about 0.06%, or less than about 0.05% Compound 7 after the formulation is stored at 2-8° C. or at about 25° C./60% RH for 1 month, 3 months, 6 months, or 9 months, including all values therebetween. In one embodiment, the formulation comprising Compound I, or a pharmaceutically acceptable salt, ester, and/or solvate thereof, comprises less than about 1%, less than about 0.5%, less than about 0.4%, less than about 0.3%, less than about 0.2%, less than about 0.15%, less than about 0.1%, less than about 0.09%, less than about 0.08%, less than about 0.07%, less than about 0.06%, or less than about 0.05% Compound 7 after the formulation is stored at 2-8° C. or at about 25° C./60% RH for 12 months, 18 months or 24 months, including all values therebetween. In one embodiment, the formulation comprising Compound I, or a pharmaceutically acceptable salt, ester, and/or solvate thereof, comprises less than about 0.1%, less than about 0.09%, less than about 0.08%, or less than about 0.07% Compound 7 after the formulation is stored at 2-8° C. or at about 25° C./60% RH for 12 months, 18 months or 24 months, including all values therebetween.

In one embodiment, the formulation comprising Compound I, or a pharmaceutically acceptable salt, ester, and/or solvate thereof, comprises less than about 1%, less than about 0.5%, less than about 0.4%, less than about 0.3%, less than about 0.2%, less than about 0.15%, less than about 0.1%, less than about 0.09%, less than about 0.08%, or less than about 0.07% impurities after the formulation is stored at 2-8° C. or at about 25° C./60% RH for 12 months, 18 months, or 24 months, including all values therebetween.

In some embodiments, the purity and impurities of the formulation comprising Compound I are measured as area % by high-performance liquid chromatography (HPLC).

In one embodiment, a liquid formulation can be for intravenous administration.

Liquid pharmaceutical compositions may further comprise solid excipients where the components are dissolved or suspended in a liquid carrier such as water, vegetable oil, alcohol, polyethylene glycol, propylene glycol, or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Preservatives and chelating agents such as alcohol, sodium benzoate, butylated hydroxyl toluene, butylated hydroxyanisole, and ethylenediamine tetraacetic acid may be added at levels safe for ingestion to improve storage stability.

A liquid composition may also contain a buffer such as gluconic acid, lactic acid, citric acid or acetic acid, sodium gluconate, sodium lactate, sodium citrate, or sodium acetate. Selection of excipients and the amounts used may be readily determined by the formulation scientist based upon experience and consideration of standard procedures and reference works in the field.

In one embodiment, a liquid formulation comprises Compound I, or a pharmaceutically acceptable salt and/or solvate thereof, at a concentration greater than about 10 mg/mL, greater than about 11 mg/mL, greater than about 12 mg/mL, greater than about 13 mg/mL, greater than about 14 mg/mL, greater than about 15 mg/mL, greater than about 16 mg/mL, greater than about 17 mg/mL, greater than about 18 mg/mL, greater than about 19 mg/mL, greater than about 20 mg/mL, greater than about 21 mg/mL, greater than about 22 mg/mL, greater than about 23 mg/mL, greater than about 24 mg/mL, greater than about 25 mg/mL, greater than about 26 mg/mL, greater than about 27 mg/mL, greater than about 28 mg/mL, greater than about 29 mg/mL, greater than about 30 mg/mL, greater than about 31 mg/mL, greater than about 32 mg/mL, greater than about 33 mg/mL, greater than about 34 mg/mL, greater than about 35 mg/mL, or any other value or range of values therein.

In some embodiments, the formulation comprising Compound I, or a pharmaceutically acceptable salt and/or solvate thereof, at a concentration of about 15 mg/mL, about 20 mg/mL, 25 mg/mL, 30 mg/mL, or 35 mg/mL, including all values therebetween. In some embodiments, the formulation comprises Compound I, or a pharmaceutically acceptable salt and/or solvate thereof, at a concentration of about 30 mg/mL. In one embodiment, the formulation comprising Compound I, or a pharmaceutically acceptable salt and/or solvate thereof, at a concentration of about 15 mg/mL to about 35 mg/mL is further diluted prior to administration via infusion or intravenous (IV) injection.

In one embodiment, the formulation comprising Compound I, or a pharmaceutically acceptable salt and/or solvate thereof, is prepared in vials. In some embodiments, the vial containing the formulation is substantially free of aluminum. In some embodiments, the vial containing the formulation is substantially free of aluminum on its interior surface. In some embodiments, the vial containing the formulation has an inert coating on its interior surface. In some embodiments, the vial containing the formulation has SiO₂ coating on its interior surface. In some embodiments, the vial containing the formulation has a polymer coating on its interior surface. In some embodiments, the vial containing the formulation is sulfur-treated on its interior surface. In one embodiment, the vial is a glass vial.

In some embodiments, the stopper or the seal for the vial containing the formulation is substantially free of aluminum. In some embodiments, the stopper or the seal for the vial containing the formulation is substantially free of aluminum on its interior surface. In some embodiments, the stopper or the seal for the vial containing the formulation has an inert coating on its interior surface.

A formulation of the present invention for single use may contain Compound I or a pharmaceutically acceptable salt or ester thereof, in an amount of about 5 mg to about 500 mg, or any value in between. In one embodiment, the formulation comprises Compound I or a pharmaceutically acceptable salt or ester thereof in an amount of about: 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 175 mg, 180 mg, 190 mg, 200 mg, 210 mg, 220 mg, 225 mg, 230 mg, 240 mg, 250 mg, 260 mg, 270 mg, 275 mg, 280 mg, 290 mg, 300 mg, 310 mg, 320 mg, 325 mg, 330 mg, 340 mg, 350 mg, 360 mg, 370 mg, 375 mg, 380 mg, 390 mg, 400 mg, 410 mg, 420 mg, 425 mg, 430 mg, 440 mg, 450 mg, 460 mg, 470 mg, 475 mg, 480 mg, 490 mg, or 500 mg.

Therapeutic Use

The present invention also provides treatment of disorders related to proliferation of cells, comprising administering any one of the formulations comprising Compound I, or a pharmaceutically acceptable salt, ester, and/or solvate thereof, as disclosed herein. In one embodiment, a method for selectively activating p53 protein comprising contacting a cell afflicted by disorder related to cell proliferation is provided, comprising administering any one of the formulation comprising Compound I, or a pharmaceutically acceptable salt, ester, and/or solvate thereof, as disclosed herein. In one embodiment, the method comprises contacting cancer and/or tumor cells with any one of the formulations disclosed herein. In another embodiment, the method of contacting cancer and/or tumor cells with any one of the formulations disclosed herein, may induce cell apoptosis or alleviate or prevent the progression of the disorder.

In one embodiment, the present invention provides a method for stabilizing G-quadruplex (G4) comprising contacting a cell afflicted by disorder related to cell proliferation with a formulation comprising Compound I, or a pharmaceutically acceptable salt, ester, and/or solvate thereof, as disclosed herein. In one embodiment, the method comprises contacting cancer and/or tumor cells with any one of the formulations disclosed herein. In another embodiment, the method of contacting cancer and/or tumor cells with any one of the formulations disclosed herein, may induce cell apoptosis or alleviate or delay the progression of the disorder.

In one embodiment, a formulation comprising Compound I, or a pharmaceutically acceptable salt, ester, and/or solvate thereof, can be administered in an amount effective to stabilize G4 in cancer and/or tumor cells, which may lead to cell death or apoptosis.

The present invention also provides methods of treating, preventing, ameliorating and/or alleviating the progression of disorders or conditions characterized by cell proliferation in a subject. More particularly, the methods of the present invention involve administration of an effective amount of formulation comprising Compound I, or a pharmaceutically acceptable salt, ester, and/or solvate thereof, in a subject to treat a disorder or a condition characterized by cell proliferation. The formulation comprising Compound I, or a pharmaceutically acceptable salt, ester, and/or solvate thereof, can be administered in an amount effective selectively activate p53 proteins in cancer and/or tumor cells, which may lead to cell death or apoptosis. The terms “subject” and “patient” are used interchangeably throughout the present application. In one embodiment, the present invention relates to method of treating cancer comprising administering to a subject in need thereof an effective amount of any one of the formulation comprising Compound I, or a pharmaceutically acceptable salt, ester, and/or solvate thereof, as disclosed herein. In one embodiment, cancer treated or ameliorated by the method as disclosed herein may be selected from Acute Lymphoblastic Leukemia, Acute Myeloid Leukemia, Adrenocortical Carcinoma, AIDS-Related Cancers, Kaposi Sarcoma, Lymphoma, Anal Cancer, Appendix Cancer, Astrocytomas, Childhood Atypical Teratoid/Rhabdoid Tumor, Basal Cell Carcinoma, Skin Cancer (Nonmelanoma), Childhood Bile Duct Cancer, Extrahepatic Bladder Cancer, Ewing Sarcoma Family of Tumors, Malignant Fibrous Histiocytoma, Brain Stem Glioma, Brain Tumors, Embryonal Tumors, Germ Cell Tumors, Craniopharyngioma, Ependymoma, Bronchial Tumors, Burkitt Lymphoma (Non-Hodgkin Lymphoma), Carcinoid Tumor, Gastrointestinal Carcinoma of Unknown Primary, Cardiac (Heart) Tumors, Lymphoma, Cervical Cancer, Childhood Cancers, Chordoma, Chronic Lymphocytic Leukemia, Chronic Myelogenous Leukemia, Chronic Myeloproliferative Neoplasms Colon Cancer, Colorectal Cancer, Cutaneous T-Cell Lymphoma, Ductal Carcinoma In Situ, Endometrial Cancer, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Intraocular Melanoma, Retinoblastoma, Gallbladder Cancer, Gastric (Stomach) Cancer, Gastrointestinal Carcinoid Tumor, Gastrointestinal Stromal Tumors, Extragonadal Cancer, Ovarian Cancer, Testicular Cancer, Gestational Trophoblastic Disease, Glioma, Brain Stem Cancer, Hairy Cell Leukemia, Head and Neck Cancer, Heart Cancer, Hepatocellular (Liver) Cancer, Histiocytosis, Langerhans Cell Cancer, Hodgkin Lymphoma, Hypopharyngeal Cancer, Kidney Cancer, Renal Cell Cancer, Wilms Tumor and Other Childhood Kidney Tumors, Langerhans Cell Histiocytosis, Laryngeal Cancer, Leukemia, Chronic Lymphocytic Cancer, Chronic Myelogenous Cancer, Hairy Cell Cancer, Lip and Oral Cavity Cancer, Liver Cancer (Primary), Lobular Carcinoma In Situ (LCIS), Lung Cancer, Non-Small Cell Cancer, Small Cell Cancer, Hodgkin Cancer, Non-Hodgkin Cancer, Macroglobulinemia, Waldenström, Male Breast Cancer, Malignant Fibrous Histiocytoma of Bone and Osteosarcoma, Melanoma, Intraocular (Eye) Cancer, Merkel Cell Carcinoma, Mesothelioma, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic Myeloid Leukemia, Acute Multiple Myeloma, Chronic Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and Oropharyngeal Cancer, Epithelial Cancer, Low Malignant Potential Tumor, Pancreatic Cancer, Pancreatic Neuroendocrine Tumors (Islet Cell Tumors), Papillomatosis, Paraganglioma, Parathyroid Cancer, Penile Cancer, Pharyngeal Cancer, Pheochromocytoma, Pituitary Tumor, Pleuropulmonary Blastoma, Primary Central Nervous System Lymphoma, Rectal Cancer, Rhabdomyosarcoma, Salivary Gland Cancer, Sarcoma, Osteosarcoma (Bone Cancer), Soft Tissue Cancer, Uterine Cancer, Sezary Syndrome, Childhood Melanoma, Nonmelanoma, Small Cell Lung Cancer, Small Intestine Cancer, Soft Tissue Sarcoma, Squamous Cell Carcinoma, Childhood Squamous Neck Cancer with Occult Primary, Metastatic Cancer, T-Cell Lymphoma, Cutaneous Cancer, Throat Cancer, Thymoma and Thymic Carcinoma, Thyroid Cancer, Transitional Cell Cancer of the Renal Pelvis and Ureter, Carcinoma of Childhood, Unusual Cancers of Childhood, Urethral Cancer, Uterine Cancer, Uterine Sarcoma, Vaginal Cancer, Vulvar Cancer, or Women's Cancers.

Additionally, disclosed are methods for treating cancers, cancer cells, tumors, or tumor cells. Non limiting examples of cancer that may be treated by the methods of this disclosure include cancer or cancer cells of colorectum, breast, lung, liver, pancreas, lymph node, colon, prostate, brain, head and neck, skin, ovary, cervical, thyroid, bladder, kidney, and blood and heart (e.g., leukemia, lymphoma, and carcinoma). Non limiting examples of tumors that may be treated by the methods of this disclosure include tumors and tumor cells of: colorectum, breast, lung, liver, pancreas, lymph node, colon, prostate, brain, head and neck, skin, kidney, and blood and heart (e.g., leukemia, lymphoma, and carcinoma), uterine, gastrointestine, larynx, and oral cavity.

In one embodiment, cancer treated or ameliorated by any one of the methods as disclosed herein may be selected from the group consisting of: heme cancer (hematologic malignancies), colorectum cancer, breast cancer, lung cancer, liver cancer, ovarian cancer, cervical cancer, Ewing's sarcoma, pancreatic cancer, cancer of the lymph nodes, colon cancer, prostate cancer, brain cancer, cancer of the head and neck, skin cancer, kidney cancer, cancer of the heart, uterine cancer, gastrointestinal malignancies, and carcinomas of the larynx and oral cavity. In some embodiments, the cancer treated or ameliorated by the method is selected from the group consisting of uterine cancer, gastrointestinal malignancies, and carcinomas of the larynx and oral cavity. In one embodiment, cancer treated or ameliorated by the method is hematologic malignancies which is selected from the group consisting of: leukemia, lymphoma, myeloma, and multiple myeloma. In one embodiment, cancer treated or ameliorated by any one of the methods as disclosed herein may be selected from the group consisting of hematologic malignancies, colorectal cancer, breast cancer, lung cancer, liver cancer, ovarian cancer, cervical cancer, Ewing's sarcoma, pancreatic cancer, cancer of the lymph nodes, colon cancer, prostate cancer, brain cancer, cancer of the head and neck, skin cancer, kidney cancer, osteosarcoma, and cancer of the heart. In one embodiment, cancer treated or ameliorated by the method is heme cancer which is selected from the group consisting of: leukemia, lymphoma, myeloma, and multiple myeloma.

In one embodiment, any one of the formulations comprising Compound I, or a pharmaceutically acceptable salt, ester, and/or solvate thereof, disclosed herein can be useful for treating breast cancer. In one embodiment, the formulation is useful for treating ovarian cancer. In one embodiment, the formulation is useful for treating solid tumors. In one embodiment, the formulation is useful for treating pancreatic cancer. In one embodiment, the formulation is useful for treating pancreatic tumor. In one embodiment, the formulation is useful for treating non-small cell lung cancer. In one embodiment, the formulation is useful for treating hematologic malignancies. In one embodiment, the formulation is useful for treating hematologic malignancies.

In one embodiment, cancer treated or ameliorated by any one of the methods as disclosed herein can be wherein the subject has a mutation in a DNA repair gene. In a specific embodiment, the DNA repair gene is a homologous recombinant gene. In another embodiment, the DNA repair gene is a gene in the homologous recombination (HR) dependent deoxyribonucleic acid (DNA) double strand break (DSB) repair pathway. In a specific embodiment, the DNA repair gene is a homologous recombinant (HR) or non-homologous end joining (NHEJ) gene. In another embodiment, the DNA repair gene is a gene in the homologous recombination (HR) or non-homologous end joining (NHEJ) dependent deoxyribonucleic acid (DNA) double strand break (DSB) repair pathway. In another method, the DNA repair gene is one or more genes selected from the group consisting of BRCA1, BRCA2, ATM, ATR, CHK1, CHK2, Rad51, RPA and XRCC3.

In one embodiment of any one of the methods as disclosed herein, the subject has a mutation in one or more genes in the HR pathway, Fanconi anemia pathway, mismatch repair pathway, ATM pathway, cell cycle pathway, p53 signaling pathway, polymerase pathway, topoisomerase pathway. In one embodiment, the subject has a mutation in one or more genes having a function in HR repair, ATM pathway, cell cycle, topoisomerase, double-strand break repair, excision repair, C-Myb transcription factor network, p-53 signaling, and/or apoptosis or genomic stability. In one embodiment, the subject has a mutation in one or more genes selected from BRCA1, BRCA2, PTEN, ATM, CHEK1, TOP2A, ABL1, PER1, RAD51, ERCC5, NBN, TRIM28, SETMAR, RAD54L, EYA1, and TP53. In one embodiment, the subject has a mutation in one or more genes selected from ARID1A, ATM, ATR, BAP1, BARD1, BLM, BRCA1, BRCA2, CHEK1, CHEK2, ERCC3, FANCG, FANCI, FANCL, HELQ, MLH1, MRE11A, MSH2, MSH6, MUTYH, PMS1, POLE, POLR1B, PTEN, RAD17, RAD51D, RAD54L, TOP3A, and/or WRN.

In one embodiment, the subject has a mutation in one or more genes selected from BRCA1, BRCA2, TP53, and PALB2. In another embodiment, the subject has a mutation in BRCA1, and/or BRCA2 genes, and/or other genes of the HR pathway. In some embodiments, the mutation is a somatic mutation. In other embodiments, the mutation is a germline mutation.

In one embodiment, Compound I or a pharmaceutically acceptable salt thereof's efficacy is associated with a mutation or a copy number loss of a gene in the HR pathway or the Fanconi anemia pathway, wherein the gene is selected from: ARID1A, ATM, ATR, BAP1, BARD1, BLM, BRCA1, BRCA2, FANCG, FANCI, FANCL, HELQ, MRE11A, NBN, PALB2, PTEN, RAD51, RAD51D, RAD54L, and/or WRN. In one embodiment, Compound I or a pharmaceutically acceptable salt thereof's efficacy is associated with a mutation or a copy number loss of HR pathway gene BRCA2 and/or PALB2.

In another embodiment, cancer treated or ameliorated by the method comprises cancer cells harboring defects in BRCA1 gene (breast cancer type 1), BRCA2 (breast cancer type 2), and/or other members of the homologous recombination pathway. BRCA1 and BRCA2 are tumor suppressor genes, and encode proteins involved in DNA damage repair. Mutations that alter expression or activity of the BRCA1 or BRCA2 proteins may lead to the accumulation of genetic alterations in a cell, and can lead to cancer in a subject. Such mutations are referred to herein as “disease-associated mutations.”

In another embodiment, the cancer cells are deficient in BRCA1 and/or BRCA2. In another embodiment, the cancer cells are homozygous for a mutation in BRCA1 and/or BRCA2. In another embodiment, the cancer cells are heterozygous for a mutation in BRCA1 and/or BRCA2. In some embodiments, the cancer cells are deficient in germline BRCA1 and/or BRCA2. In another embodiment, the cancer cells are deficient in somatic BRCA1 and/or BRCA2.

In one embodiment, cancer treated or ameliorated by any one of the methods as disclosed herein is characterized by one or more disease-associated mutations in BRCA1 or BRCA2. In some embodiments, cancer is characterized by one or more disease-associated mutations in BRCA1 and BRCA2. In some embodiments, cancer is characterized by one or more disease-associated mutations in BRCA1 but harbors no disease-associated mutations in BRCA2. In some embodiments, cancer is characterized by one or more disease-associated mutations in BRCA2 but harbors no disease-associated mutations in BRCA1. In some embodiments, cancer is characterized by one or more disease-associated mutations in BRCA1 or BRCA2.

In one embodiment, cancer treated or ameliorated by any one of the methods as disclosed herein is BRCA2 deficient. In another embodiment, Compound I or a pharmaceutically acceptable salt or solvate thereof or the compound of the present invention induces more apoptotic cell death in BRCA2 deficient or BRCA2 knockout cells relative to BRCA2 proficient or BRCA2 wild type cells. In one embodiment, Compound I or a pharmaceutically acceptable salt or solvate thereof or the compound of the present invention is selectively toxic to BRCA2 deficient or BRCA2 knockout cells over BRCA2 proficient or BRCA2 wild type cells. In other embodiments, BRCA2 deficient or BRCA2 knockout cells exhibit higher sensitivity to Compound I or a pharmaceutically acceptable salt or solvate thereof or the compound of the present invention as compared to BRCA2 proficient or BRCA2 wild type cells.

In one embodiment, cancer treated or ameliorated by any one of the methods as disclosed herein is BRCA mutant or BRCA-like mutant cancer. In some embodiments, the BRCA mutant or BRCA-like mutant cancer is a BRCA2-mutated cancer. In other embodiments, the BRCA mutant or BRCA-like mutant cancer is breast cancer, ovarian cancer, pancreatic cancer, or prostate cancer. In one embodiment, the BRCA mutant or BRCA-like mutant cancer is breast cancer or prostate cancer. In one embodiment, cancer treated or ameliorated by any one of the methods as disclosed herein is BRCA mutant cancer. In some embodiments, the BRCA mutant cancer is a BRCA2-mutated cancer. In other embodiments, the BRCA mutant cancer is breast cancer, ovarian cancer, pancreatic cancer, or prostate cancer. In other embodiments, the BRCA mutant cancer is breast cancer, ovarian cancer, or pancreatic cancer. In one embodiment, the BRCA mutant cancer is breast cancer or prostate cancer.

In one embodiment, cancer treated or ameliorated by any one of the methods as disclosed herein is BRCA-driven cancer. In some embodiments, cancer is BRCA-1 or BRCA2-driven cancer.

In one embodiment, the present disclosure relates to methods for treating or ameliorating cell proliferation disorder in a human subject, comprising administering to a subject in need thereof a therapeutically effective amount of a compound of the invention or a formulation prepared from a compound of the present invention as disclosed herein. In some embodiments, the human subject carries a BRCA mutation. In other embodiments, the human subject carries a BRCA2 mutation. In another embodiment, the human subject is homozygous for a mutation in BRCA2.

In one embodiment, the present disclosure relates to methods for treating or ameliorating cell proliferation disorder in a human subject, comprising administering to a subject in need thereof a therapeutically effective amount of a compound of the invention or a formulation prepared from a compound of the present invention. In some embodiments, the human subject carries a BRCA mutation. In other embodiments, the human subject carries a BRCA2 mutation. In another embodiment, the human subject is homozygous for a mutation in BRCA2.

In one embodiment, the BRCA2 mutation is substitution, deleterious truncating, splicing, insertion or deletion of BRCA2 gene. In some embodiments, the BRCA2 mutation is a loss-of-function mutation.

In one embodiment, BRCA2 mutation exists as a coding change or mutation in one or more of 4088insA, c.68-80insT, c.793+34T>G, 999del5, 6503delTT, 4486delG, 2594delC, 5382insC, 3829delT, Q563X, 3438G>T, 1675delA, 999del5, 8295T4A, 9900insA, 5579insA, 7647delTG, 7253delAA, 9303ins31, 3034del4 bp, 5910C3G, 6676insTA, 6085G>T, 8765delAG, 3398delAAAAG, 1499insA, 7525_7526insT, 6174delT, c.289G>T, c.2950G>T, c.7963C>T, c.8878C>T, IVS6p1G4A, 6503-6504delTT, 9132delC, 9254del5, c.9254_9258delATCAT, c.3492_3493insT, 9475A>G, c.9026_9030delATCAT, c.3264insT, c.8978_8991del14, c.156_157insAlu, 6238ins2del21, 10323delCins11, 8876delC, 8138_8142del5, c.8765_8766delAG, exons 21-24 del, c.6589delA, 4817A>G, 8477delAGA, 8984delG, G4X, 3783del10, c.5101C>T, c.5433_5436delGGAA, c.7806-2A>G, c.5291C>G, c.3975_3978dupTGCT, IVS16-2A>G, c.3318C>A, c.4790C>A, 9326insA and 6174delT, 8984delG, 1913T>A, 1342C>A, 3199A>G, 1093A>C, c.3394C>T, c.7697T>C, 5531delTT, C5507G, 6174delT, c.5373_5376 del GTAT, c.373G>T, S2219X, C1290Y, 6633del5, 3034delACAA, 818delA, exons 8-9 del, c.3036_3039delACAA, c.6024_6025_delTA, c.2732_2733insA, c.3870_3873delG, 4150G>T, 6027del4, c.5114_5117delTAAA, c.2639_2640delTG, 6880 insG, 3034 del AAAC, 695insT, 1528del4, 9318del4, S1099X, 5802delAATT, 8732C>A, c.2835C>A, c.7480C>T, 1627A.T, 3972delTGAG, 7708C.T, 7883delTTAA, c.2808_2811delACAA, c.3109C>T, c.7436_7805del370, c.9097_9098insA, 2670delC, 3073delT, 6696-7delTC, exons 4-11 dup, 4859delA, 4265delCT, 1342C.A, 490 delCT, 3337C>T, 5057delTG, g.-1235G>A, g.-26G>A, g.681+56C>T, c.865A>C, c.1114A>C, c.1365A>G, c.2229T>C, c.2971A>G, c.3396A>G, c.3516G>A, c.3807T>C, c.4415_4418delAGAA, c.5529A>C, c.6033_6034insGT, c.7242A>G, g.7435+53C>T, g.7806-14T>C, g.8755-66T>C, c.4415-4418delAGAA, c.6033insGT, c.5576_5579delTTAA, c.9485-1G>A, 4265delCT, 4859delA, 6775G>T, p.Glu2183X, c.2699_2704delTAAATG, 4706delAAAG, R2336P, IVS2+1G>A, 8765delAG, 999 del 5, 1537 del4, 5909 insA, c.211dupA, c.3381delT/3609delT, c.7110delA/7338delA, c.7235insG/7463insG, c.2826_2829del, c.6447_6448dup, c.5771_5774del, and/or 5999del4. See Karami, F. et al. BioMed Res. Int'l. 2013, 2013, Article ID 928562, which is hereby incorporated by reference in its entirety for all purposes.

In one embodiment, BRCA2 mutation exists as a coding change or mutation in one or more of c.8537_8538del AG, c.8537_8538del AG mutation in exon 20, c.859G>C, c. 859G>C in exon 7, c.4614T>C, p.Ser1538Ser synonymous mutation, c.5946delT, p.S1982fs, c.6819DelinsGT, c.6592G>T, c.3847_3848delGT, c.6821G>T, or c.6821G>T in exon 11.

In one embodiment, the compound of the present disclosure demonstrate sensitivity to a BRCA2 null cell line relative to the parental cell line. In one embodiment, the sensitivity of the BRCA2 null cell line is at least two hundred-fold greater than the BRCA2 wild type cell line. In other embodiments, the sensitivity is at least twenty-fold higher. In some embodiments, the sensitivity is at least 200-fold higher. In other embodiments, the sensitivity is at least 2, 5, 10, 15, 20, 25, 30, 35, 40, 45, 50, 60, 70, 80, 90, 100, 125, 150, 175, 200 or 400-fold higher.

In one embodiment, the present disclosure relates to methods for treating cancer in a subject, comprising administering a therapeutically effective amount of any one of the formulation comprising Compound I, or a pharmaceutically acceptable salt and/or solvate thereof to the subject, as disclosed herein, wherein the subject has a PALB2 mutation and/or a BRCA2 mutation. In one embodiment, the subject has a PALB2 mutation. In one embodiment, the subject has a BRCA2 mutation. In one embodiment, the subject has a PALB2 mutation and a BRCA2 mutation. In one embodiment, the subject has one or more additional gene mutation in the homologous recombination pathway.

In another embodiment, cancer treated or ameliorated by the method comprises cancer cells harboring defects in PALB2 gene. In another embodiment, the cancer cells are deficient in PALB2. In another embodiment, the cancer cells are homozygous for a mutation in PALB2. In another embodiment, the cancer cells are heterozygous for a mutation in PALB2.

In one embodiment, Compound I or a pharmaceutically acceptable salt or solvate thereof or the compound of the present invention induces more apoptotic cell death in PALB2 deficient or PALB2 knockout cells relative to PALB2 proficient or PALB2 wild type cells. In one embodiment, Compound I or a pharmaceutically acceptable salt or solvate thereof or the compound of the present invention is selectively toxic to PALB2 deficient or PALB2 knockout cells over PALB2 proficient or PALB2 wild type cells. In other embodiments, PALB2 deficient or PALB2 knockout cells exhibit higher sensitivity to Compound I or a pharmaceutically acceptable salt or solvate thereof or the compound of the present invention as compared to PALB2 proficient or PALB2 wild type cells.

In one embodiment, cancer treated or ameliorated by any one of the methods as disclosed herein is PALB2 mutant or PALB2-like mutant cancer. In some embodiments, the PALB2 mutant or PALB2-like mutant cancer is a PALB2-mutated cancer. In other embodiments, the PALB2 mutant or PALB2-like mutant cancer is breast cancer, ovarian cancer, pancreatic cancer, or prostate cancer. In one embodiment, the PALB2 mutant or PALB2-like mutant cancer is breast cancer or prostate cancer. In one embodiment, cancer treated or ameliorated by any one of the methods as disclosed herein is PALB2 mutant cancer (PALB2-mutated cancer). In other embodiments, the PALB2 mutant cancer is breast cancer, ovarian cancer, pancreatic cancer, or prostate cancer. In other embodiments, the PALB2 mutant cancer is breast cancer, ovarian cancer, or pancreatic cancer. In one embodiment, the PALB2 mutant cancer is breast cancer or prostate cancer.

In one embodiment, the PALB2 mutation is a loss-of-function mutation of the PALB2 gene. In one embodiment, the PALB2 mutation causes PALB2 gene to lose its function. In one embodiment, the PALB2 mutation is substitution, deleterious truncating, splicing, insertion or deletion of PALB2 gene. In some embodiments, the PALB2 mutation is a monoallelic loss-of-function mutation. In other embodiments, the PALB2 mutation is a biallelic loss-of-function mutation.

In one embodiment, the present disclosure relates to a method for treating or ameliorating cell proliferation disorder in a human subject, comprising administering to a subject in need thereof a therapeutically effective amount of a compound of the invention or a formulation prepared from a compound of the present invention as disclosed herein. In some embodiments, the human subject carries a PALB2 mutation. In another embodiment, the human subject is homozygous for a mutation in PALB2.

In one embodiment, the present disclosure relates to a method for treating or ameliorating cell proliferation disorder in a human subject, comprising administering to a subject in need thereof a therapeutically effective amount of a compound of the invention or a formulation prepared from a compound of the present invention. In some embodiments, the human subject carries a PALB2 mutation. In another embodiment, the human subject is homozygous for a mutation in PALB2.

In one embodiment, PALB2 mutation exists as a coding change in one or more of c.48G>A, c.72del, c.156del, c.172_175del, c.196C>T, c.229del, c.451C>T, c.509_510del, c.757_758del, c.886del, c.956_962del, c.1027C>T, c.1037_1041del, c.1108C>T, c.1240C>T, c.1314del, c.1431del, c.1571C>G, c.1591_1600del, c.1592del, c.1653T>A, c.2074C>T, c.2167_2168del, c.2257C>T, c.2323C>T, c.2386G>T, c.2515-1G>T, c.2521del, c.2686dup, c.2718G>A, c.2787_2788del, c.2834+1G>T, c.2835-1G>C, c.2888del, c.2919_2920del, c.2982dup, c.3022del, c.3113G>A, c.3116del, c.3201+1G>C, c.3323del, c.3423_3426del, c.3426dup, c.3456dup, c.3497_3498del, c.3504_3505del, c.3549C>A, c.3549C>G, del5340 bp, or c.3362del. See Antoniou, A. C. et al. N. Engl. J. Med. 2014, 371, 497-506, which is hereby incorporated by reference in its entirety for all purposes.

Additionally, the present disclosure relates to methods for treating cancers, cancer cells, tumors, or tumor cells comprising administering a therapeutically effective amount of any one of the formulations comprising Compound I, or a pharmaceutically acceptable salt, ester, and/or solvate thereof, disclosed herein. The present disclosure also relates to methods for treating cancers, cancer cells, tumors, or tumor cells comprising administering a therapeutically effective amount of any one of the formulations disclosed herein, to a subject in need thereof. Non limiting examples of cancer that may be treated by the methods of this disclosure include cancer or cancer cells of: colorectum, breast, ovary, cervix, lung, liver, pancreas, lymph node, colon, prostate, brain, head and neck, skin, kidney, osteosarcoma, bone (e.g., Ewing's sarcoma), blood and heart (e.g., leukemia, lymphoma, carcinoma), uterine, gastrointestinal malignancies, and carcinomas of the larynx and oral cavity. Non limiting examples of tumors that may be treated by the methods of this disclosure include tumors and tumor cells of: colorectum, breast, ovary, cervix, lung, liver, pancreas, lymph node, colon, prostate, brain, head and neck, skin, kidney, osteosarcoma, bone (e.g., Ewing's sarcoma), blood and heart (e.g., leukemia, lymphoma, carcinoma), uterine, gastrointestinal malignancies, and carcinomas of the larynx and oral cavity.

The present invention also provides methods of decreasing Pol I transcription comprising administering any one of the formulations comprising Compound I, or a pharmaceutically acceptable salt, ester, and/or solvate thereof, disclosed herein, to a subject in need. In some embodiments, the inhibition of Pol I transcription is in peripheral blood mononuclear cells (PBMC). In other embodiments, the inhibition of Pol I transcription can be observed in PBMC at one hour post-IV infusion of a dose comprising an effective amount of any one of the formulations as disclosed herein.

In one embodiment, the inhibition of Pol I transcription in PBMC 1 hour post-infusion is at an average level of about 15% inhibition or greater. In another embodiment, the Pol I transcription in PBMC 1 hour post-infusion is at an average level of about 5% inhibition or greater, about 10% inhibition or greater, about 15% inhibition or greater, about 20% inhibition or greater, about 25% inhibition or greater, about 30% inhibition or greater, about 35% inhibition or greater, about 40% inhibition or greater, about 45% inhibition or greater, about 50% inhibition or greater, about 55% inhibition or greater, about 65% inhibition or greater, or about 70% inhibition or greater.

In one embodiment of the present methods disclosed herein, the inhibition of Pol I transcription can be observed in MACS (magnetic-activated cell sorting) sorted tumor cells.

As used herein, administering can be effected or performed using any of the various methods known to those skilled in the art. Any one of the formulation comprising Compound I, or a pharmaceutically acceptable salt, ester, and/or solvate thereof, disclosed herein, can be administered, for example, subcutaneously, intravenously, parenterally, intraperitoneally, intradermally, intramuscularly, topically, enteral (e.g., orally), rectally, nasally, buccally, sublingually, vaginally, by inhalation spray, by drug pump or via an implanted reservoir in dosage formulations containing conventional non-toxic, physiologically acceptable carriers or vehicles. A formulation or a composition comprising the compound of the present invention can be administered, for example, subcutaneously, intravenously, parenterally, intraperitoneally, intradermally, intramuscularly, topically, enteral (e.g., orally), rectally, nasally, buccally, sublingually, vaginally, by inhalation spray, by drug pump or via an implanted reservoir in dosage formulations containing conventional non-toxic, physiologically acceptable carriers or vehicles. In one embodiment, the formulation of the present disclosure is administered intravenously.

Further, the formulation can be administered to a localized area in need of treatment. For example, a formulation prepared from a compound of the present invention can be administered to a localized area in need of treatment. Administration to a localized area can be achieved by, for example, and not by way of limitation, local infusion during surgery, topical application, transdermal patches, by injection, by catheter, by suppository, or by implant (the implant can optionally be of a porous, non-porous, or gelatinous material), including membranes, such as sialastic membranes or fibers.

The formulation of the present invention for administration will depend in part on the route by which it is administered. For example, for mucosal (e.g., oral mucosa, rectal, intestinal mucosa, bronchial mucosa) administration, nose drops, aerosols, inhalants, nebulizers, eye drops or suppositories can be used. The formulation of the present disclosure can be administered together with other biologically active agents, such as anticancer agents, analgesics, anti-inflammatory agents, anesthetics and other agents which can control one or more symptoms or causes of a disorder or a condition characterized by cell proliferation.

In one embodiment, the formulation of the present invention, as disclosed herein, can be administered in combination with one or more therapeutically active agent. In one embodiment, the one or more therapeutically active agent is an anticancer agent. In some embodiments, the one or more therapeutically active anticancer agents include, but are not limited to, paclitaxel, vinblastine, vincristine, etoposide, doxorubicin, hercepztin, lapatinib, gefitinib, erlotinib, tamoxifen, fulvestrant, anastrazole, lectrozole, exemestane, fadrozole, cyclophosphamide, taxotere, melphalan, chlorambucil, mechlorethamine, chlorambucil, phenylalanine, mustard, cyclophosphamide, ifosfamide, carmustine (BCNU), lomustine (CCNU), streptozotocin, busulfan, thiotepa, cisplatin, carboplatin, dactinomycin (actinomycin D), doxorubici(adriamycin), daunorubicin, idarubicin, mitoxantrone, plicamycin, mitomycin C, bleomycin, combinations thereof, and the like. In another embodiment, the one or more therapeutically active anticancer agents include, but are not limited to, PARP (poly (DP-ribose)polymerase) inhibitors. Suitable PARP inhibitors include, but are not limited to, 4-(3-(1-(cyclopropanecarbonyl)piperazine-4-carbonyl)-4-fluorobenzyl)phthalazin-1(2H)-one (Olaparib, AZD2281, Ku-0059436), 2-[(2R)-2-methylpyrrolidin-2-yl]-1H-benzimidazole-4-carboxamide (Veliparib, ABT-888), (8S,9R)-5-fluoro-8-(4-fluorophenyl)-9-(1-methyl-1H-1,2,4-triazol-5-yl)-8,9-dihydro-2H-pyrido[4,3,2-de]phthalazin-3(7H)-one (Talazoparib, BMN 673), 4-iodo-3-nitrobenzamide (Iniparib, BSI-201), 8-fluoro-5-(4-((methylamino)methyl)phenyl)-3,4-dihydro-2H-azepino[5,4,3-cd]indol-1(6H)-one phosphoric acid (Rucaparib, AG-014699, PF-01367338), 2-[4-[(dimethylamino)methyl]phenyl]-5,6-dihydroimidazo[4,5,1-jk][1,4]benzodiazepin-7(4H)-one (AG14361), 3-aminobenzamide (INO-1001), 2-(2-fluoro-4-((S)-pyrrolidin-2-yl)phenyl)-3H-benzo[d]imidazole-4-carboxamide (A-966492), N-(5,6-dihydro-6-oxo-2-phenanthridinyl)-2-acetamide hydrochloride (PJ34, PJ34 HCl), MK-4827, 3,4-dihydro-4-oxo-3,4-dihydro-4-oxo-N-[(1S)-1-phenylethyl]-2-quinazolinepropanamide (ME0328), 5-(2-oxo-2-phenylethoxy)-1(2H)-isoquinolinone (UPF-1069), 4-[[4-fluoro-3-[(4-methoxy-1-piperidinyl)carbonyl]phenyl]methyl]-1(2H)-phthalazinone (AZD 2461), 5-((3-chlorophenyl)amino)benzo[c][2,6]naphthyridine-8-carboxylic acid, and the like. In another embodiment, the one or more therapeutically active agent is an immunotherapeutic agent. In some embodiments, the one or more immunotherapeutic agents includes, but are not limited to, a monoclonal antibody, an immune effector cell, adoptive cell transfer, an immunotoxin, a vaccine, a cytokine, and the like.

In one embodiment, the one or more therapeutically active agent is selected from an alkylating agent, an anti-metabolite, a vinca alkaloid, a taxane, a topoisomerase inhibitor, an anti-tumor antibiotic, a tyrosine kinase inhibitor, an immunosuppressive macrolide, an Akt inhibitor, an HDAC inhibitor an Hsp90 inhibitor, an mTOR inhibitor, a PI3K/mTOR inhibitor, a PI3K inhibitor, a CDK (cyclin-dependent kinase) inhibitor, CHK (checkpoint kinase) inhibitor, PARP (poly (DP-ribose)polymerase) inhibitors, or combinations thereof.

In one embodiment, the one or more therapeutically active agent is a PI3K inhibitor. In another embodiment, the PI3K inhibitor is Idelalisib.

In one embodiment, the one or more therapeutically active agent is a PARP inhibitor. In another embodiment, the PARP inhibitor is Olaparib.

In other embodiments, the one or more therapeutically active agent is an agent that induces immune checkpoint blockade, such as PD-1 blockade and CTLA-4 blockade.

In some embodiments, the one or more therapeutically active agent is an antibody or an antigen-binding portion thereof that disrupts the interaction between Programmed Death-1 (PD-1) and Programmed Death Ligand-1 (PD-L1). In one embodiment, the one or more therapeutically active agent is selected from the group consisting of: an anti-PD-1 antibody, a PD-1 antagonist, an anti-PD-L1 antibody, a siRNA targeting expression of PD-1, a siRNA targeting the expression of PD-L1, and a peptide, fragment, dominant negative form, or soluble form of PD-1 or PD-L1.

In one embodiment, the one or more therapeutically active agent is a monoclonal antibody. In one embodiment, the monoclonal antibody is selected from the group consisting of anti-PD-1 antibody, nivolumab, pembrolizumab alemtuzumab, bevacizumab, brentuximab vedotin, cetuximab, gemtuzumab ozogamicin, ibritumomab tiuxetan, ipilimumab, ofatumumab, panitumumab, rituximab, tositumomab, trastuzumab, anti-B7-H4, anti-B7-H1, anti-LAG3, BTLA, anti-Tim3, anti-B7-DC, anti-CD160, MR antagonist antibodies, anti-4-1BB, anti-OX40, anti-CD27, and/or CD40 agonist antibodies. In some embodiments, the one or more therapeutically active agent is an anti-PD-1 antibody. In other embodiments, an anti-PD-1 antibody is a humanized antibody. In one embodiment, the monoclonal antibody is selected from the group consisting of nivolumab and pembrolizumab. In a specific embodiment, the monoclonal antibody is nivolumab.

In some embodiments, one or more therapeutically active agent disclosed in WO 2017/087235 is hereby incorporated by reference in its entirety for all purposes.

In another embodiment, any one of the formulations comprising Compound I, or a pharmaceutically acceptable salt, ester, and/or solvate thereof, as disclosed herein, can be administered in combination with radiotherapy.

Additionally, administration can comprise administering to the subject a plurality of dosages over a suitable period of time. Such administration regimens can be determined according to routine methods, upon a review of the instant disclosure.

Compound I or a pharmaceutically acceptable salt and/or solvate is generally administered in a dose of about 0.01 mg/kg/dose to about 100 mg/kg/dose. Alternately the dose can be from about 0.1 mg/kg/dose to about 10 mg/kg/dose; or about 1 mg/kg/dose to 10 mg/kg/dose. Time release preparations may be employed or the dose may be administered in as many divided doses as is convenient. When other methods are used (e.g. intravenous administration), crystalline forms are administered to the affected tissue at a rate from about 0.05 to about 10 mg/kg/hour, alternately from about 0.1 to about 1 mg/kg/hour. Such rates are easily maintained when these crystalline forms are intravenously administered as discussed herein. Generally, topically administered formulations are administered in a dose of about 0.5 mg/kg/dose to about 10 mg/kg/dose range. Alternately, topical formulations are administered at a dose of about 1 mg/kg/dose to about 7.5 mg/kg/dose or even about 1 mg/kg/dose to about 5 mg/kg/dose.

A range of from about 0.1 to about 100 mg/kg is appropriate for a single dose. Continuous administration is appropriate in the range of about 0.05 to about 10 mg/kg.

Drug doses can also be given in milligrams per square meter of body surface area rather than body weight, as this method achieves a good correlation to certain metabolic and excretionary functions. Moreover, body surface area can be used as a common denominator for drug dosage in adults and children as well as in different animal species (Freireich et al., (1966) Cancer Chemother Rep. 50, 219-244). Briefly, to express a mg/kg dose in any given species as the equivalent mg/sq m dose, the dosage is multiplied by the appropriate km factor. In an adult human, 100 mg/kg is equivalent to 100 mg/kg×37 kg/sq m=3700 mg/m².

Any one of the formulations of the present invention may be administered to a patient as a daily dose either in a single dose or in divided portions served multiple times a day, such as twice, three times, or four times a day.

In one embodiment, any one of the formulation as disclosed herein is administered in a dose of about 1 mg of Compound I or a pharmaceutically acceptable salt and/or solvate thereof per body surface area of the subject (mg/m²) to about 2,000 mg/m², or any value or subranges therebetween, of Compound I, or a pharmaceutically acceptable salt and/or solvate thereof. In one embodiment, the formulation of the present invention is administered in a dose of about 25 mg/m² to about 2,000 mg/m², or any value or subranges therebetween, of Compound I, or a pharmaceutically acceptable salt and/or solvate thereof. In one embodiment, the formulation of the present invention can be administered in a dose of about 25 mg/m², about 30 mg/m², about 35 mg/m², about 40 mg/m², about 45 mg/m², about 50 mg/m², about 55 mg/m², about 60 mg/m², about 65 mg/m², about 70 mg/m², about 75 mg/m², about 80 mg/m², about 85 mg/m², about 90 mg/m², about 95 mg/m², about 100 mg/m², about 110 mg/m², about 120 mg/m², about 125 mg/m², about 130 mg/m², about 140 mg/m², about 150 mg/m², about 160 mg/m², about 170 mg/m², about 175 mg/m², about 180 mg/m², about 190 mg/m², about 200 mg/m², about 210 mg/m², about 220 mg/m², about 225 mg/m², about 230 mg/m², about 240 mg/m², about 250 mg/m², about 260 mg/m², about 270 mg/m², about 275 mg/m², about 280 mg/m², about 290 mg/m², about 300 mg/m², about 310 mg/m², about 320 mg/m², about 325 mg/m², about 330 mg/m², about 340 mg/m², about 350 mg/m², about 360 mg/m², about 370 mg/m², about 375 mg/m², about 380 mg/m², about 390 mg/m², about 400 mg/m², about 410 mg/m², about 420 mg/m², about 425 mg/m², about 430 mg/m², about 440 mg/m², about 450 mg/m², about 460 mg/m², about 470 mg/m², about 475 mg/m², about 480 mg/m², about 490 mg/m², about 500 mg/m², about 510 mg/m², about 520 mg/m², about 525 mg/m², about 530 mg/m², about 540 mg/m², about 550 mg/m², about 560 mg/m², about 570 mg/m², about 575 mg/m², about 580 mg/m², about 590 mg/m², about 600 mg/m², about 610 mg/m², about 620 mg/m², about 625 mg/m², about 630 mg/m², about 640 mg/m², about 650 mg/m², about 660 mg/m², about 670 mg/m², about 675 mg/m², about 680 mg/m², about 690 mg/m², about 700 mg/m², about 710 mg/m², about 720 mg/m², about 725 mg/m², about 730 mg/m², about 740 mg/m², about 750 mg/m², about 760 mg/m², about 770 mg/m², about 775 mg/m², about 780 mg/m², about 790 mg/m², about 800 mg/m², about 810 mg/m², about 820 mg/m², about 825 mg/m², about 830 mg/m², about 840 mg/m², about 850 mg/m², about 860 mg/m², about 870 mg/m², about 875 mg/m², about 880 mg/m², about 890 mg/m², about 900 mg/m², about 910 mg/m², about 920 mg/m², about 925 mg/m², about 930 mg/m², about 940 mg/m², about 950 mg/m², about 960 mg/m², about 970 mg/m², about 975 mg/m², about 980 mg/m², about 990 mg/m², about 1000 mg/m², or any value in between, of Compound I, or a pharmaceutically acceptable salt and/or solvate thereof. In one embodiment, the formulation of the present invention can be administered in a dose of about 50 mg, about 100 mg, about 150 mg, about 170 mg, about 325 mg, about 475 mg, or about 650 mg of Compound I or a pharmaceutically acceptable salt and/or solvate thereof. In some embodiments, the dose can vary depending on the health of the patients or the patient's sensitivity to Compound I, or a pharmaceutically acceptable salt and/or solvate thereof.

In one embodiment, the formulation of the present invention is administered in a dose of about 50 mg/m² to about 800 mg/m², or any value or subranges therebetween, of Compound I, or a pharmaceutically acceptable salt and/or solvate thereof. In one embodiment, the formulation of the present invention is administered in a dose of about 50 mg/m² to about 650 mg/m², or any value or subranges therebetween, of Compound I, or a pharmaceutically acceptable salt and/or solvate thereof. In one embodiment, the formulation of the present invention is administered in a dose of about 100 mg/m² to about 700 mg/m², or any value or subranges therebetween, of Compound I, or a pharmaceutically acceptable salt and/or solvate thereof. In one embodiment, the formulation of the present invention is administered in a dose of about 150 mg/m² to about 700 mg/m², or any value or subranges therebetween, of Compound I, or a pharmaceutically acceptable salt and/or solvate thereof. In one embodiment, the formulation of the present invention is administered in a dose of about 150 mg/m² to about 650 mg/m², or any value or subranges therebetween, of Compound I, or a pharmaceutically acceptable salt and/or solvate thereof. In one embodiment, the formulation of the present invention is administered in a dose of about 250 mg/m² to about 700 mg/m², or any value or subranges therebetween, of Compound I, or a pharmaceutically acceptable salt and/or solvate thereof. In one embodiment, the formulation of the present invention is administered in a dose of about 300 mg/m² to about 700 mg/m², or any value or subranges therebetween, of Compound I, or a pharmaceutically acceptable salt and/or solvate thereof. In one embodiment, the formulation of the present invention is administered in a dose of about 400 mg/m² to about 700 mg/m², or any value or subranges therebetween, of Compound I, or a pharmaceutically acceptable salt and/or solvate thereof. In one embodiment, the formulation of the present invention is administered in a dose of about 425 mg/m² to about 675 mg/m², or any value or subranges therebetween, of Compound I, or a pharmaceutically acceptable salt and/or solvate thereof. In one embodiment, the formulation of the present invention is administered in a dose of about 450 mg/m² to about 650 mg/m², or any value or subranges therebetween, of Compound I, or a pharmaceutically acceptable salt and/or solvate thereof. In one embodiment, the formulation of the present invention is administered in a dose of about 475 mg/m² of Compound I, or a pharmaceutically acceptable salt and/or solvate thereof.

In one embodiment, any one of the formulations of the present invention is administered in a dose of about 150 mg/m² to about 300 mg/m², or any value or subranges therebetween, of Compound I, or a pharmaceutically acceptable salt and/or solvate thereof. In one embodiment, the formulation of the present invention is administered in a dose of about 150 mg/m² to about 250 mg/m², or any value or subranges therebetween, of Compound I, or a pharmaceutically acceptable salt and/or solvate thereof. In one embodiment, the formulation of the present invention is administered in a dose of about 170 mg/m² of Compound I, or a pharmaceutically acceptable salt and/or solvate thereof.

In one embodiment, any one of the formulations of the present invention can be generally administered in a dose of about less than about 500 mg/m² of Compound I, or a pharmaceutically acceptable salt and/or solvate thereof. In another embodiment, the formulation of the present invention are generally administered in a dose of less than about 500 mg/m², less than about 490 mg/m², less than about 480 mg/m², less than about 475 mg/m², less than about 470 mg/m², less than about 460 mg/m², less than about 450 mg/m², less than about 440 mg/m², less than about 430 mg/m², less than about 420 mg/m², less than about 410 mg/m², less than about 400 mg/m², less than about 390 mg/m², less than about 380 mg/m², less than about 375 mg/m², less than about 370 mg/m², less than about 360 mg/m², less than about 350 mg/m², less than about 340 mg/m², less than about 330 mg/m², less than about 320 mg/m², less than about 310 mg/m², less than about 300 mg/m², less than about 290 mg/m², less than about 280 mg/m², less than about 275 mg/m², less than about 270 mg/m², less than about 260 mg/m², less than about 250 mg/m², less than about 240 mg/m², less than about 230 mg/m², less than about 220 mg/m², less than about 210 mg/m², less than about 200 mg/m², less than about 190 mg/m², less than about 180 mg/m², or less than about 170 mg/m², or any value in between, of Compound I, or a pharmaceutically acceptable salt and/or solvate thereof.

In some embodiments, any one of the formulation of the present invention can be administered to a cancer patient in a dose of less than about 750 mg/m², less than about 700 mg/m², less than about 600 mg/m², less than about 500 mg/m², less than about 475 mg/m², less than about 400 mg/m², less than about 325 mg/m², less than about 300 mg/m², less than about 200 mg/m², less than about 170 mg/m², or any subranges therein, of Compound I, or a pharmaceutically acceptable salt and/or solvate thereof. In other embodiments, the formulation of the present invention can be administered to a cancer patient in a dose of less than about 170 mg/m² of Compound I, or a pharmaceutically acceptable salt and/or solvate thereof, every three weeks. In one embodiment, the cancer patient is a heme cancer patient.

In some embodiments, any one of the formulation of the present invention can be administered to a cancer patient in about 50 mg/m² to about 1,550 mg/m², about 150 mg/m² to about 1,250 mg/m², about 250 mg/m² to about 1,050 mg/m², about 350 mg/m² to about 950 mg/m², about 375 mg/m² to about 850 mg/m², about 425 mg/m² to about 850 mg/m², about 450 mg/m² to about 800 mg/m², or about 500 mg/m² to about 750 mg/m², or any subranges therein, of Compound I, or a pharmaceutically acceptable salt and/or solvate thereof. In some embodiments, the formulation of the present invention can be administered to a cancer patient in a dose of less than about 750 mg/m² of Compound I, or a pharmaceutically acceptable salt and/or solvate thereof. In other embodiments, the formulation of the present invention can be administered to a cancer patient in any of the dosing frequency, dosing cycle or dosing regimen described herein. In one embodiment, the treatment is for solid tumors.

In some embodiments, any one of the formulations of the present invention can be administered to a cancer patient at a dose of greater than about 50 mg/m² to provide clinical results including partial response, stable disease (no tumor growth), or tumor shrinkage. In some embodiments, compounds of the present invention or formulation prepared by compounds of the present invention can be administered to a cancer patient at a dose of greater than about 100 mg/m² to provide clinical results including partial response, stable disease, or tumor shrinkage. In some embodiments, the formulation of the present invention can be administered to a cancer patient at a dose of greater than about 150 mg/m² to provide clinical results including partial response, stable disease, or tumor shrinkage.

The formulation of the present invention may be administered, hourly, daily, weekly, or monthly. The formulation of the present invention may be administered twice a day or once a day. The formulation of the present invention may be administered with food or without food.

In one embodiment, any one of the formulations of the present invention, is administered once a week, once every two weeks, once every three weeks, once every four weeks, or once a month. In some embodiments, the formulation of the present invention, is administered in a four-week treatment cycle comprising one administration weekly (QW×4). In some embodiments, the formulation of the present invention, is administered in a four-week treatment cycle comprising one administration weekly for two weeks followed by two weeks of rest period (no treatment) (QW×2). In some embodiments, the administration is on a four-week treatment cycle comprising one administration weekly for three weeks followed by one week of rest period (no treatment). In some embodiments, the formulation of the present invention, is administered in a three-week treatment cycle comprising one administration weekly for two weeks followed by one week of rest period. In another embodiment, the formulation of the present invention, is administered once every three weeks. In other embodiments, the formulation of the present invention, is administered once every three weeks by IV infusion.

In some embodiments, the treatment regimen with any one of the formulations comprising Compound I, or a pharmaceutically acceptable salt and/or solvate thereof, as disclosed herein, can last from 1 cycle to 20 cycles or greater period of time. An appropriate length of the treatment can be determined by a physician.

In some embodiments, the treatment with the formulation of the invention results in PK ranges as disclosed in PCT/US2019/018225, the disclosures of which are hereby incorporated by reference in their entireties for all purposes.

In settings of a gradually progressive disorder or condition characterized by cell proliferation, the formulation of the present application is generally administered on an ongoing basis. In certain settings administration of any one of the formulations disclosed herein can commence prior to the development of disease symptoms as part of a strategy to delay or prevent the disease. In other settings any one of the formulations disclosed herein is administered after the onset of disease symptoms as part of a strategy to slow or reverse the disease process and/or part of a strategy to improve cellular function and reduce symptoms.

It will be appreciated by one of skill in the art that dosage range should be large enough to produce the desired effect in which the neurodegenerative or other disorder and the symptoms associated therewith are ameliorated and/or survival of the cells is achieved, but not be so large as to cause unmanageable adverse side effects. It will be understood, however, that the specific dose level for any particular patient will depend on a variety of factors including the activity of the specific crystalline form employed; the age, body weight, general health, sex and diet of the individual being treated; the time and route of administration; the rate of excretion; other drugs which have previously been administered; and the severity of the particular disease undergoing therapy, as is well understood by those skilled in the art. The dosage can also be adjusted by the individual physician in the event of any complication. No unacceptable toxicological effects are expected when crystalline forms disclosed herein are used in accordance with the present application.

An effective amount any one of the formulations as disclosed herein comprises Compound I, or a pharmaceutically acceptable salt and/or solvate thereof, in amounts sufficient to produce a measurable biological response. Actual dosage levels of Compound I, or a pharmaceutically acceptable salt and/or solvate thereof, in the formulation of the present disclosure can be varied so as to administer an amount of Compound I, or a pharmaceutically acceptable salt and/or solvate thereof, effective to achieve the desired therapeutic response for a particular subject and/or application. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to medical professionals.

Further with respect to the methods of the present application, a preferred subject is a vertebrate subject. A preferred vertebrate is warm-blooded; a preferred warm-blooded vertebrate is a mammal. The subject treated by the presently disclosed methods is desirably a human, although it is to be understood that the principles of the present application indicate effectiveness with respect to all vertebrate species which are included in the term “subject.” In this context, a vertebrate is understood to be any vertebrate species in which treatment of a neurodegenerative disorder is desirable.

As such, the present application provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos or farms. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economical importance to humans. Thus, also provided are the treatment of livestock, including, but not limited to, domesticated swine, ruminants, ungulates, horses (including race horses), poultry, and the like.

The following examples further illustrate the present invention but should not be construed as in any way limiting its scope.

Any one of the formulations as disclosed herein can be used for any one of the methods disclosed herein, including treating cancer. Any one of the formulations as disclosed herein for Compound I or a pharmaceutically acceptable salt and/or solvate thereof can be used for any one of the methods disclosed herein, including treating cancer. Any one of the dosing schedules as disclosed herein can be used for any one of the methods disclosed herein, including treating cancer.

### EXAMPLES

### Example 1. Synthesis of Compound I

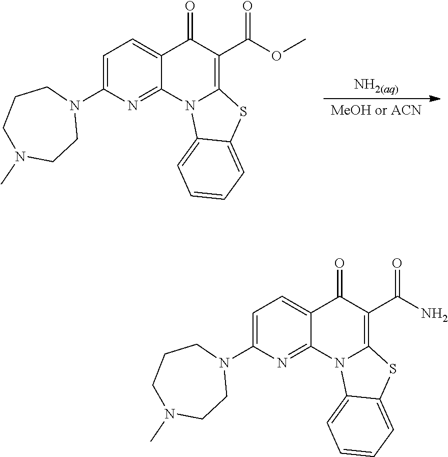

To a 5 L reactor was added ethyl 2-(4-methyl-1,4-diazepan-1-yl)-5-oxo-5H-benzo[4,5]thiazolo[3,2-a][1,8]naphthyridine-6-carboxylate (101 g, see WO 2009/046383 for synthesis) and acetonitrile (2 L). To the mixture was added 28-30% NH₃(aq) (1950 ml) then heated up to 60° C. (a lot of NH₃ gas released) with condenser temperature at −13° C. The mixture was stirred for 4 days and additional 28-30% NH3(aq) (400 ml) was added. The mixture was stirred for 2 days and additional 28-30% NH3(aq) (200 ml) was added. The mixture was stirred for 1 day and additional 28-30% NH3(aq) (100 ml) was added. After 10 days of stirring, a lot of solid precipitated. The mixture was cooled to room temperature and stirred for 1 day. The mixture was filtered to get 92 g of crude Compound I in 53.8% yield with 94.66% purity and the loss on drying (LOD) was 42.1%.

To a 2 L reactor was added 38 g of crude Compound I and 800 ml of acetonitrile. The resulting mixture was heated to 60° C. and then added 800 ml of 28-30% NH₃(aq). The mixture was stirred for 3 hr, filtrated at 50-60° C. and washed with 350 ml of acetonitrile. The wet cake was dried at 40° C. overnight to get 34.8 g of Compound I (2-(4-methyl-1,4-diazepan-1-yl)-5-oxo-5H-benzo[4,5]thiazolo[3,2-a][1,8]naphthyridine-6-carboxamide) in 92% yield with 98.0% purity and the LOD was 2.30%. MS: m/z 408.136 [M+H]⁺.

### Example 2: Preparation of Liquid Formulation of Compound I (30 mg/mL)

Composition:

| | Concentration | Concentration | Quantity |
|---|---|---|---|
| | (% w/v) | (mg/mL) | (g) |
| Compound I | 3.0 | 30.0 | 6.0 |
| HCl (1N) | As needed, for pH adjustment | | As needed |
| NaOH (1N) | As needed, for pH adjustment | | As needed |
| 0.9% Saline | Diluted to volume | Diluted to volume | Q.S. to |
| | to make 100 mL | to make 100 mL | 200 mL |

Compounding: Compound I formulation was compounded in anaerobic environment, filtered and filled in a 20 mL clear glass vial (Type I; West Pharma/Schott) with Novapure 20 mm rubber stopper (B2-TR coating), sealed with 20 mm flip-off TruEdge seals (target fill volume 5 mL) and packaged with reclosable bag and oxygen absorbents.

Nitrogen Purged Water for Injection: A 250 mL glass beaker with about 150 ml water for Injection, USP, was sparged with nitrogen for at least 30 minutes. The dissolved oxygen content was measured using Dissolved Oxygen CHEMetrics. Sparging was continued until oxygen level of 1 ppm or less was achieved. Once the desired oxygen level was achieved, the nitrogen line was removed and the headspace of the beaker was flushed, and immediately covered with parafilm.

Nitrogen Purged 0.9% Saline: A 500 mL glass bottle with about 500 mL sterile 0.9% Saline was sparged with nitrogen for at least 30 minutes while the opening of the bottle was covered with aluminum foil. The dissolved oxygen content was measured using Dissolved Oxygen CHEMetrics. Sparging was continued until oxygen level of 1 ppm or less was achieved. Once the desired oxygen level was achieved, the nitrogen line was removed and the headspace of the bottle was flushed, and the bottle was immediately covered.

All materials required for preparing the formulation was taken into the glovebox. The glovebox was purged with nitrogen for at least 30 minutes. The bottle containing Compound I was only opened once anaerobic condition was achieved. The anaerobic condition was monitored using BWC2R-X O2 Single Gas Detector with a acceptable range of 19.5% vol to 23.5% vol.

In the glovebox, under anaerobic condition, 250 mL glass bottle was weighed. 180±0.1 g of sterile 0.9% saline was added to the glass bottle. Using the sintered glass sparger, the saline in the 250 mL glass bottle was sparged with nitrogen. The dissolved oxygen content was measured using Dissolved Oxygen CHEMetrics. Sparging was continued until oxygen level of 1 ppm or less was achieved. While stirring (540 rpm), 6.04±0.01 g Compound I (adjusted based on calculated correction factor using Certificate of Analysis of Compound I sample used) was added to the bottle. The weigh boat was rinsed 3 times with approximately 2 mL of sparged sterile 0.9% saline each time. The solution was further mixed at a set speed of 540 rpm until it became clear. The mixing speed was adjusted to achieve a good vortex during mixing, if needed.

About 0.5 mL solution was removed and the pH was measured using the Oakton pH meter. If the pH was not between 4.4-4.6, pH of the solution was adjusted with 1N hydrochloric acid, NF, or 1N sodium hydroxide, NF. The sample removed for pH measurement was discarded. The pH-adjusted solution was mixed further for about 2 hours at a set speed of 540 rpm with nitrogen blanketing (nitrogen flow no more than 5 psi). The formulated solution was weighed and if there was weight loss due to evaporation, sparged sterile 0.9% saline was added to bring the final formulation to the target gross weight.

Target Gross Weight=tare weight of the bottle at the beginning+200 g−volume of solution removed for pH measurement

Once the weight was adjusted, the dissolved oxygen content was measured using Dissolved Oxygen CHEMetrics. Sparging was continued until oxygen level of 1 ppm or less was achieved. With a 50 mL sterile syringe connected to a PVDF filter, 5 mL of the filtered solution was added to each serum glass vial, capped, and sealed with an aluminum seal. Total of 22 vials were filled. Vials were transferred out from the glove box, visually inspected for appearance of solution and integrity of seal. Each vial was individually packed into 3×4″ reclosable aluminum pouch with 10 bags of oxygen absorber, purged with nitrogen and the pouch was sealed.

The pH of the solution was not checked again after stirring for 2 hours and before filtering and filling the glass vials. Later during stability study as described in Example 3, it was found that the pH of the solution had become 5.8 after the vials were filled and sealed.

### Example 3. Stability Study of Compound I Formulation

Liquid formulation of Compound I (30 prepared according to Example 2 were subjected to stability test conditions at 2-8° C. (about 5° C.) and at 25° C./60% RH for a period shown in Tables 1-2. Each 20 mL glass vial ample was individually packed into 3×4″ reclosable aluminum pouch with 10 bags of oxygen absorber, purged with nitrogen and sealed, prior to placing under the stability conditions.

At each time point, a vial was removed from the stored condition and reconstituted with 10 mL of D5W and analyzed by HPLC method described below.

RP-HPLC Chromatography Parameters (Base Mobile Phase)

| Parameter | Conditions |
|---|---|
| Column | Waters XBridge Phenyl, |
| | 150 mm (L) × 4.6 mm (ID), 3.5 μM |
| Mobile Phase A | 10 mM Na₂HPO₄, pH 11.0 |
| Mobile Phase B/ | Methanol |
| Needle Wash | |
| Diluent | 0.1% Trifluoroacetic |
| | Acid in Water |
| Injection volume | 10 μL |
| Run Time | 58 min |
| Detection Wavelength | 240 nm |

The relative retention time of certain related substance with respect to Compound I are shown below:

| Compound | RRT (RP-HPLC basic mobile phase) |
|---|---|
| 1A | 0.62 |
| 7 | 0.75 |
| 10 | 1.12 |

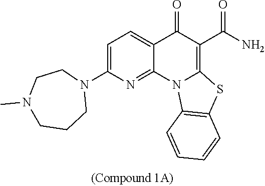 [SMILES: CN1CCCN(c2ccc3c(=O)c(C(N)=O)c4sc5ccccc5n4c3n2)CC1]

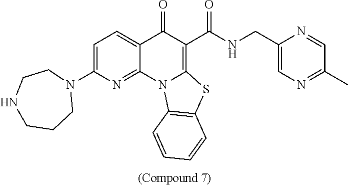 [SMILES: Cc1cnc(CNC(=O)c2c(=O)c3ccc(N4CCCNCC4)nc3n3c2sc2ccccc23)cn1]

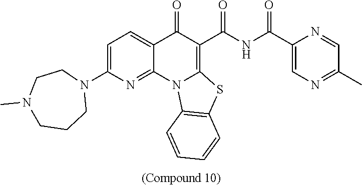

TABLE 1
Stability Assay Results at 2-8° C.

| | Initial | 1 month | 3 months | 6 months |
|---|---|---|---|---|
| | Colorless, clear, | Colorless, clear, | Colorless, clear, | Colorless, clear, |
| | no free particles | no free particles | no free particles | no free particles |
| | can be observed | can be observed | can be observed | can be observed |
| Appearance | in the solution | in the solution | in the solution | in the solution |
| pH | 5.8 | 5.8 | 5.8 | 5.8 |
| (USP <791>) | | | | |
| Compound I | 97.6% | 97.6% | 102.7% | 102.1% |
| by HPLC | | | | |
| assay | | | | |
| Related | All individual | All individual | All individual | Comp. 7 =0.05% |
| Substances | related substance | related substance | related substance | |
| | <0.05% | <0.05% | <0.05% | |
| | Total: <0.05% | Total: <0.05% | Total: <0.05% | Total: 0.05% |
| | 9 months | 12 months | 18 months | 24 months |
| | Colorless, clear, | Colorless, clear, | Colorless, clear, | Colorless, clear, |
| | no free particles | no free particles | no free particles | no free particles |
| | can be observed | can be observed | can be observed | can be observed |
| Appearance | in the solution | in the solution | in the solution | in the solution |
| pH | 5.8 | 5.8 | 5.8 | 5.8 |
| (USP <791>) | | | | |
| Compound I | 102.0% | 101.9% | 105.6% | 101.7% |
| by HPLC | | | | |
| assay | | | | |
| Related | All individual | Comp. 7 = 0.07% | All individual | Comp. 7 = 0.05% |
| Substances | related substance | | related substance | |
| | <0.05% | | <0.05% | |
| | Total: <0.05% | Total: 0.07% | Total: <0.05% | Total: 0.05% |

TABLE 2
Stability Assay Results at 25° C./60% RH

| | Initial | 1 month | 3 months | 6 months |
|---|---|---|---|---|
| | Colorless, clear, | Colorless, clear, | Colorless, clear, | Colorless, clear, |
| | no free particles | no free particles | no free particles | no free particles |
| | can be observed | can be observed | can be observed | can be observed |
| Appearance | in the solution | in the solution | in the solution | in the solution |
| pH | 5.8 | 5.8 | 5.8 | 5.8 |
| (USP <791>) | | | | |
| Compound I | 97.6% | 100.2% | 102.4% | 103.5% |
| by HPLC | | | | |
| assay | | | | |
| Related | All individual | All individual | Comp. 7 =0.05% | Comp. 7 =0.06% |
| Substances | related substance | related substance | | |
| | <0.05% | <0.05% | | |
| | Total: <0.05% | Total: <0.05% | Total: 0.05% | Total: 0.06% |
| | 9 months | 12 months | 18 months | 24 months |
| | Colorless, clear, | Colorless, clear, | Colorless, clear, | Colorless, clear, |
| | no free particles | no free particles | no free particles | no free particles |
| | can be observed | can be observed | can be observed | can be observed |
| Appearance | in the solution | in the solution | in the solution | in the solution |
| pH | 5.8 | 5.8 | 5.8 | 5.8 |
| (USP <791>) | | | | |
| Compound I | 103.9% | 101.9% | 105.0% | 100.8% |
| by HPLC | | | | |
| assay | | | | |
| Related | All individual | Comp. 7 = 0.07% | All individual | Comp. 7 = 0.05% |
| Substances | related substance | | related substance | Comp. 10 = 0.06% |
| | <0.05% | | <0.05% | |
| | Total: <0.05% | Total: 0.07% | Total: <0.05% | Total: 0.11% |

### Example 4. Comparative Stability Study of Compound I Liquid Formulation with Monosodium Phosphate Buffer

To demonstrate the superior stability results of the liquid formulation as discussed in Examples 2 and 3, comparative stability data for other Compound I liquid formulations are provided below.

Compound I was formulated into ready-to-use solution having the following composition:

| | |
|---|---|
| Compound I (non-lyophilized) | 250 mg |
| Monosodium Phosphate USP | 59.99 mg |
| 1M HC1 (to adjust pH to 6) | 0.33 mg |
| Sterile Water for Injection | Fill to 10.0 mL |

Compound I was dissolved in monosodium phosphate buffer solution, the pH was adjusted to 6 with 1M HCl, and sterile filtration was carried out. The filtered solution was filled into 10 mL vials (Type I tubing glass vial; 20 mm serum/lyophilization multi-compendial) and sealed with stoppers and flip-off seals. The preparation was not carried out under anaerobic conditions and sparging of reagents or the resulting formulation with nitrogen was not performed. Six-month stability data for this formulation with monosodium phosphate is shown in Table 3. Compared to the 6-month stability disclosed in Tables 1-2 which had total impurity of 0.05% and 0.06% by HPLC at 6-month, the formulation containing monosodium phosphate buffer had significantly greater amount of impurities at 6 months (Table 3, 1.26% and 3.91%). The formulation containing monosodium phosphate buffer already had significantly higher total impurities even before the vials were placed under stability conditions (Table 3, Initial, 1.03%).

TABLE 3
Six-Month Stability Assay Results for Compound
I Formulation with Monosodium Phosphate Buffer

| | | 6 month | 6 month |
|---|---|---|---|
| | | 25° C./60% | 40° C./75% |
| | | RH Upright | RH Upright |
| | Initial | Vial Position | Vial Position |
| Appearance | Clear, pale | Clear, pale | Clear, pale |
| | yellow | yellow | yellow |
| | solution | solution | solution |
| pH | 5.9 | 6.0 | 6.0 |
| Compound I by | 99% | 99% | 96% |
| HPLC assay (%) | | | |
| Total Impurities | 1.03 | 1.26 | 3.91 |
| by HPLC (%) | | | |
| Particulate | ≥10 μm = 263 | ≥10 μm = 2287 | ≥10 μm = 1443 |
| Matter | ≥25 μm = 7 | ≥25 μm = 134 | ≥25 μm = 101 |
| (counts/vial) | | | |

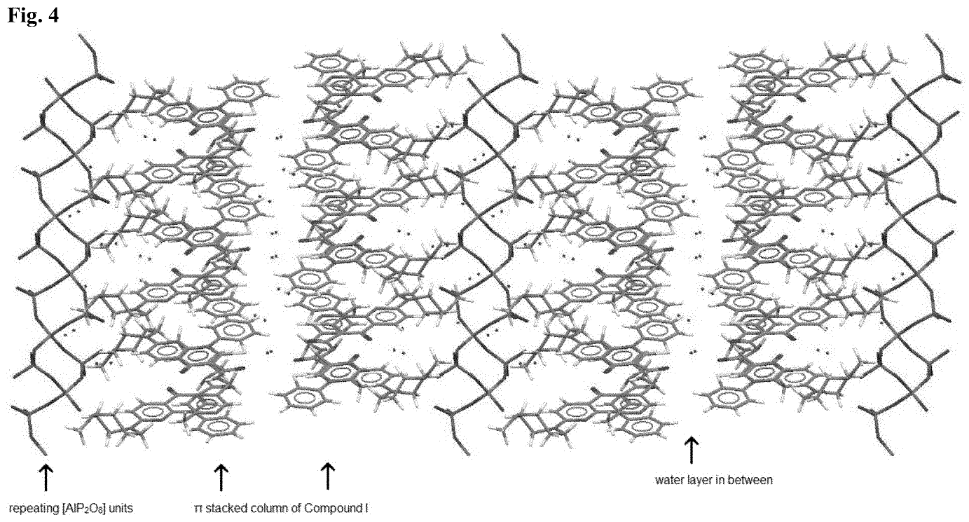

The sizing and counting of particulate matter disclosed in Table 3 was performed using the light obscuration method. The number of particulate matters increased while being stored for 6 months under the stability conditions. The crystalline material (colorless needle) obtained from the formulation with monosodium phosphate buffer was submitted for single crystal X-ray diffraction analysis. It was determined that the crystalline material found in the formulation was consistent with it being a hydrated Compound I aluminophosphate having a molecular formula C₂₇H₂₇N₇O_{12.87}Al_{0.5}PS. The structure was determined to be a hydrated crystal form, composed of two Compound I molecules, fourteen single atoms that were assigned as water molecules, and corner-shared AlO₄ and PO₄ tetrahedra that made up a one-dimensional chain of [AlP₂O₈]n. Packing diagram viewed along the b axis is shown in FIG. 4. Compound I molecules interact with each other through π-π stacking interactions down the a axis. Adjacent π stacked columns are separated from each other by layers of water and a one-dimensional chain of repeating [AlP₂O₈] units.

Without bound to any theory, the phosphate buffer and the aluminum eluting or leaching from the glass vials (borosilicate) have reacted to form the aluminophosphate complex during storage/stability study. See Ogawa et al. Chem. Pharm. Bull. 2013, 31, 539-545. Compound I likely participated in the aluminophosphate complex formation as the crystal structure confirmed the molecular structure of Compound I in a hydrated crystal form interacting with the aluminophosphate repeating units.

### Biological Assays and Examples

### Example 5. Cell Viability Assessment and Cell Proliferation Assessment

The effect of Compound I on cell viability was assessed by Alamar Blue assay of metabolic activity in various cancer cell lines. Table 4 shows Compound I demonstrate broad spectrum antiproliferative activity in multiple cancer cell lines, while being significantly less active in normal cells.

TABLE 4
Compound I EC₅₀ in Cell Viability Assay

| Cell Line | Cancer Type | EC₅₀ (nM) | Cell Line | Cancer Type | EC₅₀ (nM) |
|---|---|---|---|---|---|
| EOL-1 | Leukemia | 3 | SK-MEL-24 | Melanoma | 147 |
| SR | Leukemia | 5 | HCT-116 | Colon | 164 |
| MOLT-3 | Leukemia | 6 | NK92mi | Lymphoma | 165 |
| MV 4;11 | Leukemia | 12 | MDA-MB-468 | Breast | 171 |
| SEM | Leukemia | 18 | NCI-H2170 | Lung | 194 |
| A7 | Melanoma | 23 | U2OS | Osteosarcoma | 281 |
| NCI-H460 | Lung | 38 | BT-20 | Breast | 335 |
| THP-1 | Leukemia | 47 | MCF 7 | Breast | 347 |
| NCI-H1299 | Lung | 55 | SUM 190PT | IBC* | 583 |
| A375 | Melanoma | 58 | BxPC-3 | Pancreatic | 664 |
| Jurkat | Leukemia | 64 | HT-29 | Colon | 741 |
| Ramos | Lymphoma | 66 | SUM 149PT | IBC* | 751 |
| RPMI-8226 | Myeloma | 68 | PC-3 | Prostate | 1,100 |
| NCI-H520 | Lung | 70 | SK-MES-1 | Lung | 1,260 |
| MIA PaCa-2 | Pancreatic | 74 | Hs 578.T | Breast | 1,647 |
| SK-OV-3 | Ovarian | 78 | UACC-812 | Breast | 1,830 |
| HL60 | Leukemia | 83 | MDA-MB-361 | Breast | 2,100 |
| MDA-MB-231 | Breast | 83 | T47D | Breast | 2,337 |
| BT-474 | Breast | 86 | MDA-MB-175-VII | Breast | 2,780 |
| COLO-205 | Colon | 96 | A549 | Lung | 4,900 |
| K562 | Leukemia | 104 | Saos-2 | Osteosarcoma | 5,000 |
| Hs 605.T | Breast | 116 | PANC-1 | Pancreatic | 5,000 |
| ZR-75-1 | Breast | 123 | LNCaP | Prostate | 5,500 |
| Raji | Lymphoma | 133 | CCD-1058Sk | Normal | 4,710 |
| SKBr3 | Breast | 134 | CCD-1094Sk | Normal | 4,810 |
| MDA-MB-453 | Breast | 140 | CCD-1068Sk | Normal | 5,070 |
| Daudi | Lymphoma | 142 | BJ-hTERT | Normal | 5,174 |
| HL60/MX2 | Leukemia | 147 | CCD-1096Sk | Normal | 5,260 |

*IBC = Invasive ductal breast carcinoma (inflammatory)

### Example 6: Treatment of Cancer Harboring BRCA1 or BRCA2 Mutation with Compound I

In pharmacokinetic and dose escalation studies, wadult patients with metastatic, recurrent, locally, advanced, or unresectable solid malignancy (patients with histologically and/or cytologically confirmed solid malignancy), who received prior anti-cancer treatment(s) until disease progression, at 10 different dose levels of Compound I were evaluated. Groups 1-7 were administered Compound I intravenously on days 1 and 8 of a 4-week cycle at 50, 100, 150, 200, 250, 325, and 475 mg/m² (solid Compound I reconstituted with 5% glucose in sterile water or similar biologically acceptable IV fluid), respectively. Groups 8-10 were administered intravenously on days 1, 8, and 15 of a 4-week cycle at 325, 475, and 650 mg/m², respectively. Each patient received each dose over 1 hour by IV infusion on days described above.

18 Patients were diagnosed with metastatic breast cancer. Of the 18 patients, 10 patients with metastatic breast cancer with BRCA1/2 germline and relevant somatic mutations, who did not receive prior PARP inhibitor treatments.

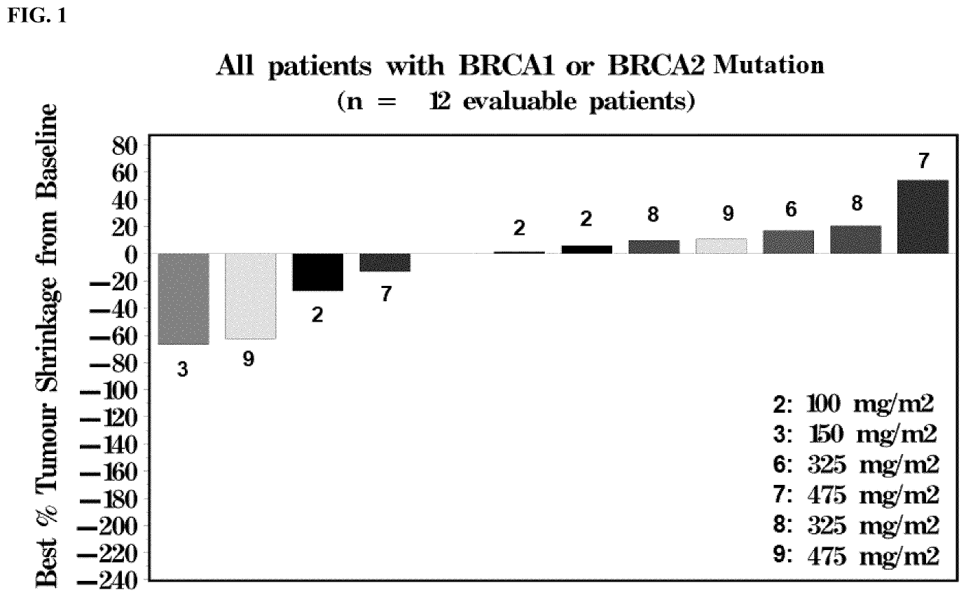

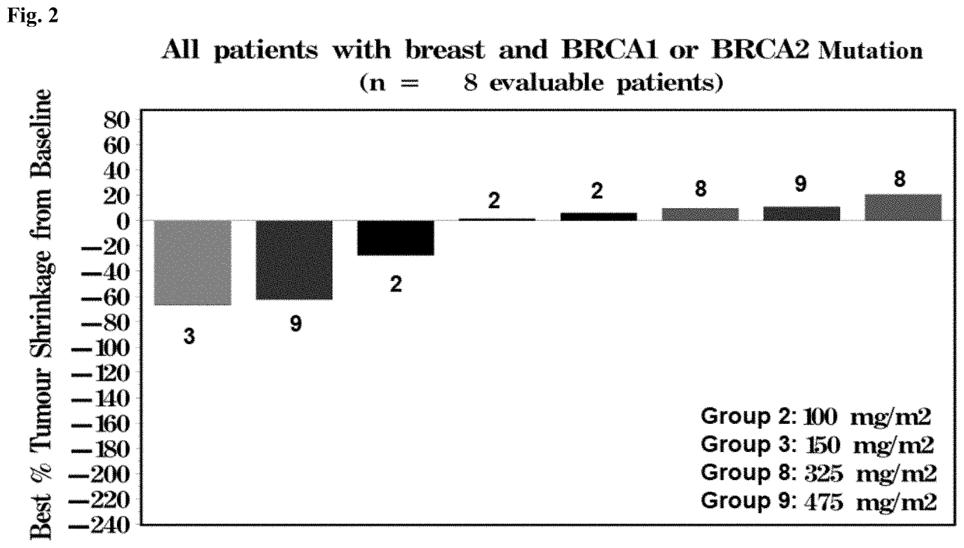

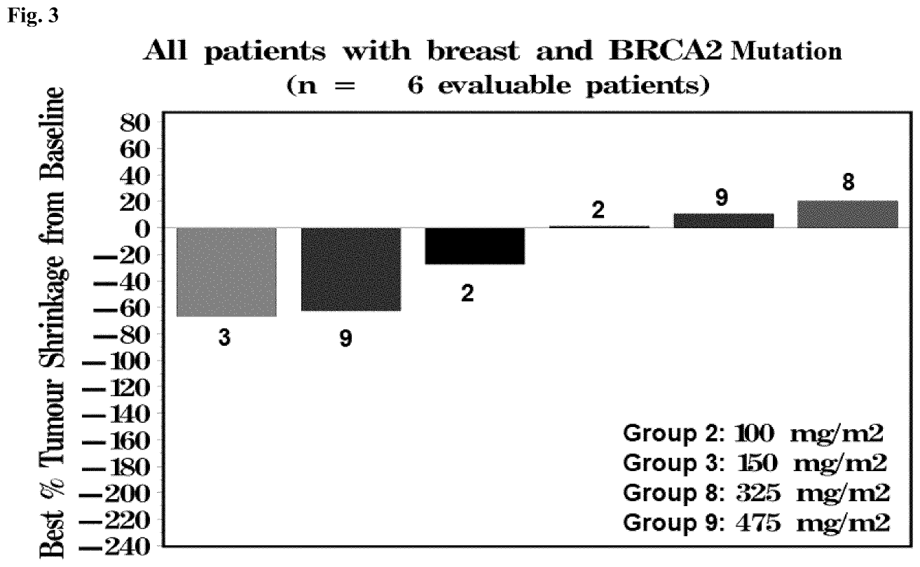

12 Patients harbored BRCA1 or BRCA2 mutation. The summary of these 12 patients' tumor size during the treatment is shown in FIG. 1. From the same group of 12 patients harboring BRCA1 or BRCA2 mutation, 8 patients had breast cancer (FIG. 2). Of the 8 patients, 6 patients harbored BRCA2 mutation and had breast cancer (FIG. 3). In FIGS. 1-3, each bar represents an individual in a treatment Group as indicated by the Group number corresponding to the treatment Groups.

One of the 10 patients enrolled in this study was from Group 10 (650 mg/m²), who harbored PALB2 mutation and BRCA2 mutation and showed partial response (PR) to Compound I treatment.

This study indicated that patients with BRCA2 mutation responded to Compound I treatment at a dose greater than or equal to 150 mg/m², where tumor shrinkage were observed.

The disclosures of all publications, patents, patent applications and published patent applications referred to herein by an identifying citation are hereby incorporated herein by reference in their entirety.

In the case of any conflict between a cited reference and this specification, the specification shall control. In describing embodiments of the present application, specific terminology is employed for the sake of clarity. However, the invention is not intended to be limited to the specific terminology so selected. Nothing in this specification should be considered as limiting the scope of the present invention. All examples presented are representative and non-limiting. The above-described embodiments may be modified or varied, without departing from the invention, as appreciated by those skilled in the art in light of the above teachings. It is therefore to be understood that, within the scope of the claims and their equivalents, the invention may be practiced otherwise than as specifically described.

1. A liquid pharmaceutical composition comprising Compound I, or a pharmaceutically acceptable salt and/or solvate thereof and a pharmaceutically acceptable carrier or excipient, 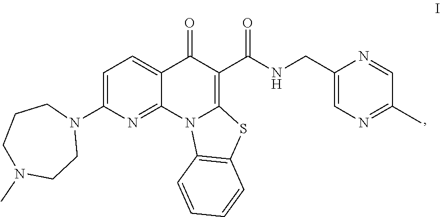
wherein the composition is substantially free of phosphates.

2. The liquid pharmaceutical composition of claim 1, wherein the composition comprises less than about 1% impurities.

3. The liquid pharmaceutical composition of claim 1, wherein the composition comprises less than about 0.5% impurities or less than about 0.15% impurities.

4. (canceled)

5. A liquid pharmaceutical composition comprising Compound I, or a pharmaceutically acceptable salt and/or solvate thereof and a pharmaceutically acceptable carrier or excipient, 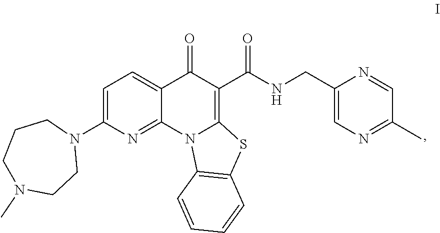
wherein the composition comprises less than about 0.1% impurities.

6.-7. (canceled)

8. The liquid pharmaceutical composition of claim 5, wherein the composition is substantially free of:
a) aluminum salts, ions, or complexes;
b) aluminophosphate;
c) a bulking agent;
d) disaccharides or sugar alcohols; or
e) sucrose, mannitol, and trehalose.

9. (canceled)

10. The liquid pharmaceutical composition of claim 8, wherein the aluminophosphate has a chain of repeating [AlP₂O₈] units.

11.-13. (canceled)

14. The liquid pharmaceutical composition of claim 5, wherein the composition comprises sterile aqueous solution and/or sterile saline solution.

15.-16. (canceled)

17. The liquid pharmaceutical composition of claim 5, wherein the composition comprises less than about 1 ppm of dissolved oxygen.

18.-21. (canceled)

22. The liquid pharmaceutical composition of claim 5, wherein the composition comprises about 0.07% or less impurities after the composition is stored at a temperature in the range of about 2° C. to about 30° C. for 12 months or for 18 months.

23. (canceled)

24. The liquid pharmaceutical composition of claim 5, wherein the composition comprises about 0.07% or less impurities after the composition is stored at a temperature in the range of about 2° C. to about 8° C. for 24 months, or the composition comprises about 0.12% or less impurities after the composition is stored at a temperature in the range of about 20° C. to about 30° C. for 24 months.

25. (canceled)

26. The liquid pharmaceutical composition of claim 5, wherein the impurity is

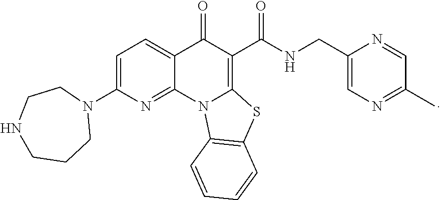 [SMILES: Cc1cnc(CNC(=O)c2c(=O)c3ccc(N4CCCNCC4)nc3n3c2sc2ccccc23)cn1]

27.-28. (canceled)

29. The liquid pharmaceutical composition of claim 5, wherein the composition is substantially free of

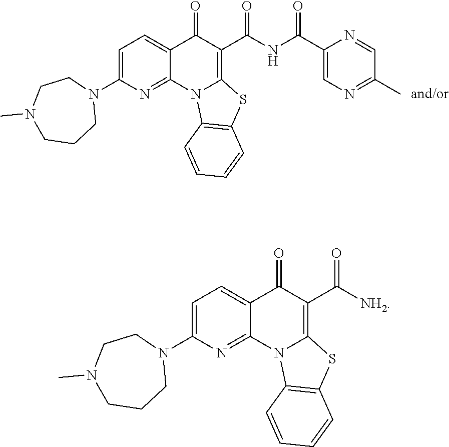

30.-32. (canceled)

33. The liquid pharmaceutical composition of claim 5, wherein the composition is substantially free of:
a) hydrated Compound I aluminophosphate complex; and/or
b) phosphate buffer.

34. (canceled)

35. The liquid pharmaceutical composition of claim 5, wherein the composition has been sparged with nitrogen to substantially remove dissolved oxygen.

36.-37. (canceled)

38. A method for treating or ameliorating cancer in a subject, comprising administering to the subject in need thereof a therapeutically effective amount of the liquid composition of claim 5.

39. (canceled)

40. The method of claim 38, wherein said cancer is heme cancer, colorectal cancer, breast cancer, lung cancer, liver cancer, ovarian cancer, cervical cancer, Ewing's sarcoma, pancreatic cancer, cancer of the lymph nodes, colon cancer, prostate cancer, brain cancer, cancer of the head and neck, bone cancer, skin cancer, kidney cancer, osteosarcoma, cancer of the heart, uterine cancer, gastrointestinal malignancies, and carcinomas of the larynx or oral cavity.

41. The method of claim 38, wherein the cancer is breast cancer, ovarian cancer, or pancreatic cancer.

42. The method of claim 40, wherein said heme cancer is leukemia, lymphoma, myeloma, or multiple myeloma.

43. The method of claim 38, wherein the subject has a mutation in a DNA repair gene.

44.-45. (canceled)

46. The method of claim 38, wherein the cancer is a BRCA-mutated or PALB2-mutated cancer.

47. (canceled)

48. The method of claim 38, wherein the cancer is characterized by one or more disease-associated mutations in BRCA1, BRCA2, or PALB2.

49.-52. (canceled)

53. A method of inhibiting Pol I transcription in a subject, comprising administering to the subject in need thereof a therapeutically effective amount of the liquid composition of claim 5.

54. (canceled)

55. A method of stabilizing G-quadruplexes (G4s) in a subject, comprising administering to the subject in need thereof a therapeutically effective amount of the liquid composition of claim 5.

56.-57. (canceled)

58. A method for treating or ameliorating cancer in a subject, comprising administering to the subject in need thereof a therapeutically effective amount of the liquid composition of claim 1.
